(19) United States
(12) Patent Application Publication
(10) Pub. No.: US 2007/0287693 A1
(43) Pub. Date: Dec. 13, 2007

(54) Benzamide Derivatives That Act Upon The Glucokinase Enzyme

(76) Inventors: Craig Johnstone, Cheshire (GB); Darren McKerrecher, Cheshire (GB); Kurt Gordon Pike, Cheshire (GB); Michael James Waring, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 11/665,222

(22) PCT Filed: Oct. 11, 2005
(86) PCT No.: PCT/GB05/03890

§ 371 Date: Apr. 12, 2007

(30) Foreign Application Priority Data
Oct. 16, 2004 (GB) 0423044.7

(51) Int. Cl. A61K 31/4155; A61P 3/10; C07D 231/40; C07D 403/12

(52) U.S. Cl. 514/210.18; 514/407; 548/364.1; 548/371.7; 548/372.5

(57) ABSTRACT

Compounds of Formula (I): 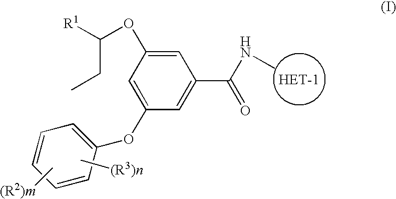
wherein R¹ is hydroxymethyl; R² is selected from —C(O)NR⁴R⁵, SO₂NR⁴R⁵, S(O)ₚR⁴ and HET-2; HET-1 is a 5- or 6-membered, optionally substituted C-linked heteroaryl ring; HET-2 is a 4-, 5- or 6-membered, C- or N-linked optionally substituted hererocyclyl ring; R³ is selected from halo, fluoromethyl, difluoromethyl, trifluoromethyl, methyl, methoxy and cyano; R⁴ is selected from, for example, hydrogen, optionally substituted (1-4C)alkyl and HET-2; R⁵ is hydrogen or (1-4C)alkyl; or R⁴ and R⁵ together with the nitrogen atom to which they are attached may form a heterocyclyl ring system as defined by HET-3; HET-3 is, for example, an optionally substituted N-linked, 4, 5 or 6 membered, saturated or partially unsaturated heterocyclyl ring; p is (independently at each occurrence) 0, 1 or 2; m is 0 or 1; n is 0, 1 or 2; provided that when m is 0, then n is 1 or 2; or a salt, pro drug or solvate thereof, are described. Their use as GLK activators, pharmaceutical compositions containing them, and processes for their preparation are also described.

The present invention relates to a group of benzoyl amino heterocyclyl compounds which are useful in the treatment or prevention of a disease or medical condition mediated through glucokinase (GLK or GK), leading to a decreased glucose threshold for insulin secretion. In addition the compounds are predicted to lower blood glucose by increasing hepatic glucose uptake. Such compounds may have utility in the treatment of Type 2 diabetes and obesity. The invention also relates to pharmaceutical compositions comprising said compounds and to methods of treatment of diseases mediated by GLK using said compounds.

In the pancreatic β-cell and liver parenchymal cells the main plasma membrane glucose transporter is GLUT2. Under physiological glucose concentrations the rate at which GLUT2 transports glucose across the membrane is not rate limiting to the overall rate of glucose uptake in these cells. The rate of glucose uptake is limited by the rate of phosphorylation of glucose to glucose-6-phosphate (G-6-P) which is catalysed by glucokinase (GLK) [1]. GLK has a high (6-10 mM) Km for glucose and is not inhibited by physiological concentrations of G-6-P [1]. GLK expression is limited to a few tissues and cell types, most notably pancreatic β-cells and liver cells (hepatocytes) [1]. In these cells GLK activity is rate limiting for glucose utilisation and therefore regulates the extent of glucose induced insulin secretion and hepatic glycogen synthesis. These processes are critical in the maintenance of whole body glucose homeostasis and both are dysfunctional in diabetes [2].

In one sub-type of diabetes, Maturity-Onset Diabetes of the Young Type 2 (MODY-2), the diabetes is caused by GLK loss of function mutations [3, 4]. Hyperglycaemia in MODY-2 patients results from defective glucose utilisation in both the pancreas and liver [5]. Defective glucose utilisation in the pancreas of MODY-2 patients results in a raised threshold for glucose stimulated insulin secretion. Conversely, rare activating mutations of GLK reduce this threshold resulting in familial hyperinsulinism [6, 6a, 7]. In addition to the reduced GLK activity observed in MODY-2 diabetics, hepatic glucokinase activity is also decreased in type 2 diabetics [8]. Importantly, global or liver selective overexpression of GLK prevents or reverses the development of the diabetic phenotype in both dietary and genetic models of the disease [9-12]. Moreover, acute treatment of type 2 diabetics with fructose improves glucose tolerance through stimulation of hepatic glucose utilisation [13]. This effect is believed to be mediated through a fructose induced increase in cytosolic GLK activity in the hepatocyte by the mechanism described below [13].

Hepatic GLK activity is inhibited through association with GLK regulatory protein (GLKRP). The GLK/GLKRP complex is stabilised by fructose-6-phosphate (F6P) binding to the GLKRP and destabilised by displacement of this sugar phosphate by fructose-1-phosphate (F1P). F1P is generated by fructokinase mediated phosphorylation of dietary fructose. Consequently, GLK/GLKRP complex integrity and hepatic GLK activity is regulated in a nutritionally dependent manner as F6P is dominant in the post-absorptive state whereas F1P predominates in the post-prandial state. In contrast to the hepatocyte, the pancreatic β-cell expresses GLK in the absence of GLKRP. Therefore, β-cell GLK activity is regulated extensively by the availability of its substrate, glucose. Small molecules may activate GLK either directly or through destabilising the GLK/GLKRP complex. The former class of compounds are predicted to stimulate glucose utilisation in both the liver and the pancreas whereas the latter are predicted to act selectively in the liver. However, compounds with either profile are predicted to be of therapeutic benefit in treating Type 2 diabetes as this disease is characterised by defective glucose utilisation in both tissues.

GLK, GLKRP and the K_{ATP} channel are expressed in neurones of the hypothalamus, a region of the brain that is important in the regulation of energy balance and the control of food intake [14-18]. These neurones have been shown to express orectic and anorectic neuropeptides [15, 19, 20] and have been assumed to be the glucose-sensing neurones within the hypothalamus that are either inhibited or excited by changes in ambient glucose concentrations [17, 19, 21, 22]. The ability of these neurones to sense changes in glucose levels is defective in a variety of genetic and experimentally induced models of obesity [23-28]. Intracerebroventricular (icv) infusion of glucose analogues, that are competitive inhibitors of glucokinase, stimulate food intake in lean rats [29, 30]. In contrast, icv infusion of glucose suppresses feeding [31]. Thus, small molecule activators of GLK may decrease food intake and weight gain through central effects on GLK. Therefore, GLK activators may be of therapeutic use in treating eating disorders, including obesity, in addition to diabetes. The hypothalamic effects will be additive or synergistic to the effects of the same compounds acting in the liver and/or pancreas in normalising glucose homeostasis, for the treatment of Type 2 diabetes. Thus the GLK/GLKRP system can be described as a potential “Diabesity” target (of benefit in both Diabetes and Obesity).

GLK is also expressed in specific entero-endocrine cells where it is believed to control the glucose sensitive secretion of the incretin peptides GIP (glucose-dependent insulinotropic polypeptide) and GLP-1 (Glucagon-Like Peptide-1) from gut K-cells and L-cells respectively (32, 33, 34). Therefore, small molecule activators of GLK may have additional beneficial effects on insulin secretion, b-cell function and survival and body weight as a consequence of stimulating GIP and GLP-1 secretion from these entero-endocrine cells.

In WO00/58293 and WO01/44216 (Roche), a series of benzylcarbamoyl compounds are described as glucokinase activators. The mechanism by which such compounds activate GLK is assessed by measuring the direct effect of such compounds in an assay in which GLK activity is linked to NADH production, which in turn is measured optically—see details of the in vitro assay described hereinafter. Compounds of the present invention may activate GLK directly or may activate GLK by inhibiting the interaction of GLKRP with GLK.

Further GLK activators have been described in WO03/095438 (substituted phenylacetamides, Roche), WO03/055482 (carboxamide and sulphonamide derivatives, Novo Nordisk), WO2004/002481 (arylcarbonyl derivatives, Novo Nordisk), and in WO03/080585 (amino-substituted benzoylaminoheterocycles, Banyu).

Our International application Number: WO03/000267 describes a group of benzoyl amino pyridyl carboxylic acids which are activators of the enzyme glucokinase (GLK).

Our International application Number: WO03/015774 describes compounds of the Formula (A): 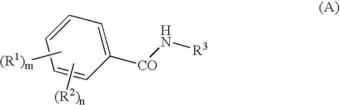
wherein R³ is a substituted heterocycle other than a carboxylic acid substituted pyridyl.

International application WO2004/076420 (Banyu) describes compounds which are generally a subset of those described in WO03/015774, wherein for example R¹ is an (substituted) alkyl ether and R² is (substituted) phenoxy.

We have surprisingly found a small group of compounds, generally a selected subgroup of those described in WO 03/015774, which have generally superior potency for the GLK enzyme, and more advantageous physical properties, including, for example, higher aqueous solubility, higher permeability, and/or lower plasma protein binding. Consequently, such compounds having a balance of these properties would be expected to display higher plasma free drug levels and superior in vivo efficacy after oral dosing as determined, for example, by activity in Oral Glucose Tolerance Tests (OGTTs). Therefore this group of compounds would be expected to provide superior oral exposure at a lower dose and thereby be particularly suitable for use in the treatment or prevention of a disease or medical condition mediated through GLK.

Thus, according to the first aspect of the invention there is provided a compound of Formula (I): 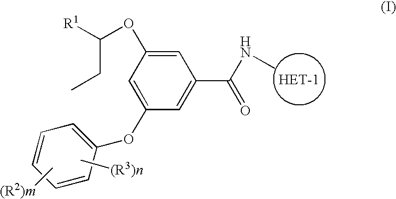
wherein:
R¹ is hydroxymethyl;
R² is selected from —C(O)NR⁴R⁵, —SO₂NR⁴R⁵, —S(O)ₚR⁴ and HET-2;
HET-1 is a 5- or 6-membered, C-linked heteroaryl ring containing a nitrogen atom in the 2-position and optionally 1 or 2 further ring heteroatoms independently selected from O, N and S; which ring is optionally substituted on an available carbon atom, or on a ring nitrogen atom provided it is not thereby quaternised, with 1 or 2 substituents independently selected from R⁶;
HET-2 is a 4-, 5- or 6-membered, C- or N-linked heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to a S(O) or S(O)₂ group, which ring is optionally substituted on an available carbon or nitrogen atom by 1 or 2 substituents independently selected from R⁷;
R³ is selected from halo, fluoromethyl, difluoromethyl, trifluoromethyl, methyl, methoxy and cyano;
R⁴ is selected from hydrogen, (1-4C)alkyl [optionally substituted by 1 or 2 substituents independently selected from HET-2, —OR⁵, —SO₂R⁵, (3-6C)cycloalkyl (optionally substituted with 1 group selected from R⁷) and —C(O)NR⁵R⁵], (3-6C)cycloalkyl (optionally substituted with 1 group selected from R⁷) and HET-2;
R⁵ is hydrogen or (1-4C)alkyl;
or R⁴ and R⁵ together with the nitrogen atom to which they are attached may form a heterocyclyl ring system as defined by HET-3;
R⁶ is independently selected from (1-4C)alkyl, halo, hydroxy(1-4C)alkyl, (1-4C)alkoxy(1-4C)alkyl, (1-4C)alkylS(O)p(1-4C)alkyl, amino(1-4C)alkyl, (1-4C)alkylamino(1-4C)alkyl, di(1-4C)alkylamino(1-4C)alkyl and HET-4;
R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
HET-3 is an N-linked, 4 to 6 membered, saturated or partially unsaturated heterocyclyl ring, optionally containing 1 or 2 further heteroatoms (in addition to the linking N atom) independently selected from O, N and S, wherein a —CH₂— group can optionally be replaced by a —C(O)— and wherein a sulphur atom in the ring may optionally be oxidised to a S(O) or S(O)₂ group; which ring is optionally substituted on an available carbon or nitrogen atom by 1 or 2 substituents independently selected from R⁸; or
HET-3 is an N-linked, 7 membered, saturated or partially unsaturated heterocyclyl ring, optionally containing 1 further heteroatom (in addition to the linking N atom) independently selected from O, S and N, wherein a —CH₂— group can optionally be replaced by a —C(O)— group and wherein a sulphur atom in the ring may optionally be oxidised to a S(O) or S(O)₂ group; which ring is optionally substituted on an available carbon or nitrogen atom by 1 or 2 substituents independently selected from hydroxy (not on nitrogen) and R⁸; or
HET-3 is an 6-10 membered bicyclic saturated or partially unsaturated heterocyclyl ring, optionally containing 1 further nitrogen atom (in addition to the linking N atom), wherein a —CH₂— group can optionally be replaced by a —C(O)—; which ring is optionally substituted on an available carbon or nitrogen atom by 1 substituent selected from R³;
R⁸ is selected from —OR⁵, (1-4C)alkyl, —C(O)(1-4C)alkyl, —C(O)NR⁴R⁵, (1-4C)alkylamino, di(1-4C)alkylamino, HET-3 (wherein said ring is unsubstituted), (1-4C)alkoxy(1-4C)alkyl, hydroxy(1-4C)alkyl and —S(O)pR⁵;
HET-4 is a 5- or 6-membered, C- or N-linked unsubstituted heteroaryl ring containing 1, 2 or 3 ring heteroatoms independently selected from O, N and S;
p is (independently at each occurrence) 0, 1 or 2;
m is 0 or 1;
n is 0, 1 or 2;
provided that when m is 0, then n is 1 or 2;
or a salt, pro-drug or solvate thereof.

In a further aspect of the invention there is provided a compound of formula (I), or a salt, pro-drug or solvate thereof as hereinbefore defined, with the proviso that compounds exemplified in WO2004/076420, which would otherwise fall within the scope of this invention, are excluded. In particular, Example numbers 19, 102, 111, 128 and 137 of WO2004/076420 are excluded.

In another aspect of the invention, there is provided a compound of the formula (I) as hereinbefore defined, wherein

R¹ is hydroxymethyl;

R² is selected from —C(O)—HET-3 and —SO₂—HET-3;

HET-1 is a 5- or 6-membered, C-linked heteroaryl ring containing a nitrogen atom in the 2-position and optionally 1 or 2 further ring heteroatoms independently selected from O, N and S; which ring is optionally substituted on an available carbon atom, or on a ring nitrogen atom provided it is not thereby quaternised, with 1 or 2 substituents independently selected from R⁶;

HET-2 is a 4-, 5- or 6-membered, C- or N-linked heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to a S(O) or S(O)₂ group, which ring is optionally substituted on an available carbon or nitrogen atom by 1 or 2 substituents independently selected from R⁷;

R³ is selected from halo, fluoromethyl, difluoromethyl, trifluoromethyl, methyl, methoxy and cyano;

R⁴ is selected from hydrogen, (1-4C)alkyl [optionally substituted by 1 or 2 substituents independently selected from HET-2, —OR⁵, —SO₂R⁵, (3-6C)cycloalkyl (optionally substituted with 1 group selected from R⁷) and —C(O)NR⁵R⁵], (3-6C)cycloalkyl (optionally substituted with 1 group selected from R⁷) and HET-2;

R⁵ is hydrogen or (1-4C)alkyl; or

R⁴ and R⁵ together with the nitrogen atom to which they are attached may form a heterocyclyl ring system as defined by HET-3;

R⁶ is independently selected from (1-4C)alkyl, halo, hydroxy(1-4C)alkyl, (1-4C)alkoxy(1-4C)alkyl, (1-4C)alkylS(O)p(1-4C)alkyl, amino(1-4C)alkyl, (1-4C)alkylamino(1-4C)alkyl, di(1-4C)alkylamino(1-4C)alkyl and HET-4;

R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;

HET-3 is an N-linked, 4, 5 or 6 membered, saturated or partially unsaturated heterocyclyl ring, optionally containing 1 or 2 further heteroatoms (in addition to the linking N atom) independently selected from O, N and S, wherein a —CH₂— group can optionally be replaced by a —C(O)— and wherein a sulphur atom in the ring may optionally be oxidised to a S(O) or S(O)₂ group; which ring is optionally substituted on an available carbon or nitrogen atom by 1 or 2 substituents independently selected from R⁸; or

HET-3 is an N-linked, 7 membered, saturated or partially unsaturated heterocyclyl ring, optionally containing 1 further heteroatom (in addition to the linking N atom) independently selected from O, S and N, wherein a —CH₂— group can optionally be replaced by a —C(O)— group and wherein a sulphur atom in the ring may optionally be oxidised to a S(O) or S(O)₂ group; which ring is optionally substituted on an available carbon or nitrogen atom by 1 or 2 substituents independently selected from R⁸; or

HET-3 is an 6-10 membered bicyclic saturated or partially unsaturated heterocyclyl ring, optionally containing 1 further nitrogen atom (in addition to the linking N atom) wherein a —CH₂— group can optionally be replaced by a —C(O)—; which ring is optionally substituted on an available carbon or nitrogen atom by 1 substituent selected from hydroxy (not on nitrogen) and R³;

R⁸ is selected from —OR⁵, (1-4C)alkyl, —C(O)(1-4C)alkyl, —C(O)NR⁴R⁵, (1-4C)alkylamino, di(1-4C)alkylamino, HET-3 (wherein said ring is unsubstituted), (1-4C)alkoxy(1-4C)alkyl, hydroxy(1-4C)alkyl and —S(O)pR⁵;

HET-4 is a 5- or 6-membered, C- or N-linked unsubstituted heteroaryl ring containing 1, 2 or 3 ring heteroatoms independently selected from O, N and S;

p is (independently at each occurrence) 0, 1 or 2;

m is 0 or 1;

n is 0, 1 or 2;

provided that when m is 0, then n is 1 or 2;

or a salt, pro-drug or solvate thereof.

In a further aspect of the invention there is provided a compound of the formula (I), as hereinbefore defined or a salt, pro-drug or solvate thereof, wherein:

HET-3 is an N-linked, 4 to 6 membered, saturated or partially unsaturated heterocyclyl ring, optionally containing 1 or 2 further heteroatoms (in addition to the linking N atom) independently selected from O, N and S, wherein a —CH₂— group can optionally be replaced by a —C(O)— and wherein a sulphur atom in the ring may optionally be oxidised to a S(O) or S(O)₂ group; which ring is optionally substituted on an available carbon or nitrogen atom by 1 or 2 substituents independently selected from R⁸.

In another aspect of the invention, there is provided a compounds of the formula (I) as hereinbefore defined, wherein

R¹ is hydroxymethyl;

R² is selected from —C(O)NR⁴¹R⁵¹, —SO₂NR⁴¹R⁵¹ and —S(O)ₚR⁴¹;

HET-1 is a 5- or 6-membered, C-linked heteroaryl ring containing a nitrogen atom in the 2-position and optionally 1 or 2 further ring heteroatoms independently selected from O, N and S; which ring is optionally substituted on an available carbon atom, or on a ring nitrogen atom provided it is not thereby quaternised, with 1 or 2 substituents independently selected from R⁶;

HET-2 is a 4-, 5- or 6-membered, C- or N-linked heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to a S(O) or S(O)₂ group, which ring is optionally substituted on an available carbon or nitrogen atom by 1 or 2 substituents independently selected from R⁷;

R³ is selected from halo, fluoromethyl, difluoromethyl, trifluoromethyl, methyl, methoxy and cyano;

R⁴¹ is selected from (1-4C)alkyl [substituted by 1 or 2 substituents independently selected from HET-2, —OR⁵, —SO₂R⁵, (3-6C)cycloalkyl (optionally substituted with 1 group selected from R⁷) and —C(O)NR⁵R⁵], (3-6C)cycloalkyl (optionally substituted with 1 group selected from R⁷) and HET-2;

R⁵¹ is hydrogen or (1-4C)alkyl;

R⁴ is selected from (1-4C)alkyl [optionally substituted by 1 or 2 substituents independently selected from HET-2, —OR⁵, —SO₂R⁵, (3-6C)cycloalkyl (optionally substituted with 1 group selected from R⁷) and —C(O)NR⁵R⁵], (3-6C)cycloalkyl (optionally substituted with 1 group selected from R⁷) and HET-2;

R⁵ is hydrogen or (1-4C)alkyl;

or R⁴ and R⁵ together with the nitrogen atom to which they are attached may form a heterocyclyl ring system as defined by HET-3;

R⁶ is independently selected from (1-4C)alkyl, halo, hydroxy(1-4C)alkyl, (1-4C)alkoxy(1-4C)alkyl, (1-4C)alkylS(O)p(1-4C)alkyl, amino(1-4C)alkyl, (1-4C)alkylamino(1-4C)alkyl, di(1-4C)alkylamino(1-4C)alkyl and HET-4;

R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;

HET-3 is an N-linked, 4, 5 or 6 membered, saturated or partially unsaturated heterocyclyl ring, optionally containing 1 or 2 further heteroatoms (in addition to the linking N atom) independently selected from O, N and S, wherein a —CH₂— group can optionally be replaced by a —C(O)— and wherein a sulphur atom in the ring may optionally be oxidised to a S(O) or S(O)₂ group; which ring is optionally substituted on an available carbon or nitrogen atom by 1 or 2 substituents independently selected from R⁸; or

HET-3 is an N-linked, 7 membered, saturated or partially unsaturated heterocyclyl ring, optionally containing 1 further heteroatom (in addition to the linking N atom) independently selected from O, S and N, wherein a —CH₂— group can optionally be replaced by a —C(O)— group and wherein a sulphur atom in the ring may optionally be oxidised to a S(O) or S(O)₂ group; which ring is optionally substituted on an available carbon or nitrogen atom by 1 or 2 substituents independently selected from R⁸; or

HET-3 is an 6-10 membered bicyclic saturated or partially unsaturated heterocyclyl ring, optionally containing 1 further nitrogen atom (in addition to the linking N atom) wherein a —CH₂— group can optionally be replaced by a —C(O)—; which ring is optionally substituted on an available carbon or nitrogen atom by 1 substituent selected from hydroxy (not on nitrogen) and R³;

R⁸ is selected from —OR⁵, (1-4C)alkyl, —C(O)(1-4C)alkyl, —C(O)NR⁴R⁵, (1-4C)alkylamino, di(1-4C)alkylamino, HET-3 (wherein said ring is unsubstituted), (1-4C)alkoxy(1-4C)alkyl, hydroxy(1-4C)alkyl and —S(O)pR⁵;

HET-4 is a 5- or 6-membered, C- or N-linked unsubstituted heteroaryl ring containing 1, 2 or 3 ring heteroatoms independently selected from O, N and S;

p is (independently at each occurrence) 0, 1 or 2;

m is 0 or 1;

n is 0, 1 or 2;

provided that when m is 0, then n is 1 or 2;

or a salt, pro-drug or solvate thereof.

In a further aspect of the invention there is provided a compound of the formula (I) as hereinbefore defined, or a salt, pro-drug or solvate thereof, wherein:

R⁴ is selected from hydrogen, (1-4C)alkyl [optionally substituted by 1 or 2 substituents independently selected from HET-2, —OR⁵, —SO₂R⁵, (3-6C)cycloalkyl (optionally substituted with 1 group selected from R⁷) and —C(O)NR⁵R⁵], and HET-2;

HET-3 as an 6-10 membered bicyclic saturated or partially unsaturated heterocyclyl ring, optionally containing 1 further nitrogen atom (in addition to the linking N atom) wherein a —CH₂— group can optionally be replaced by a —C(O)—, is optionally substituted on an available carbon or nitrogen atom by 1 substituent selected from R³.

In another aspect of the invention, there is provided a compound of the formula (I) as hereinbefore defined, wherein

R¹ is hydroxymethyl;

R² is HET-2;

HET-1 is a 5- or 6-membered, C-linked heteroaryl ring containing a nitrogen atom in the 2-position and optionally 1 or 2 further ring heteroatoms independently selected from O, N and S; which ring is optionally substituted on an available carbon atom, or on a ring nitrogen atom provided it is not thereby quaternised, with 1 or 2 substituents independently selected from R⁶;

HET-2 is a 4-, 5- or 6-membered, C- or N-linked heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to a S(O) or S(O)₂ group, which ring is optionally substituted on an available carbon or nitrogen atom by 1 or 2 substituents independently selected from R⁷;

R³ is selected from halo, fluoromethyl, difluoromethyl, trifluoromethyl, methyl, methoxy and cyano;

R⁴ is selected from hydrogen, (1-4C)alkyl [optionally substituted by 1 or 2 substituents independently selected from HET-2, —OR⁵, —SO₂R⁵, (3-6C)cycloalkyl (optionally substituted with 1 group selected from R⁷) and —C(O)NR⁵R⁵], (3-6C)cycloalkyl (optionally substituted with 1 group selected from R⁷) and HET-2;

R⁵ is hydrogen or (1-4C)alkyl;

or R⁴ and R⁵ together with the nitrogen atom to which they are attached may form a heterocyclyl ring system as defined by HET-3;

R⁶ is independently selected from (1-4C)alkyl, halo, hydroxy(1-4C)alkyl, (1-4C)alkoxy(1-4C)alkyl, (1-4C)alkylS(O)p(1-4C)alkyl, amino(1-4C)alkyl, (1-4C)alkylamino(1-4C)alkyl, di(1-4C)alkylamino(1-4C)alkyl and HET-4;

R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;

HET-3 is an N-linked, 4, 5 or 6 membered, saturated or partially unsaturated heterocyclyl ring, optionally containing 1 or 2 further heteroatoms (in addition to the linking N atom) independently selected from O, N and S, wherein a —CH₂— group can optionally be replaced by a —C(O)— and wherein a sulphur atom in the ring may optionally be oxidised to a S(O) or S(O)₂ group; which ring is optionally substituted on an available carbon or nitrogen atom by 1 or 2 substituents independently selected from R⁸; or

HET-3 is an N-linked, 7 membered, saturated or partially unsaturated heterocyclyl ring, optionally containing 1 further heteroatom (in addition to the linking N atom) independently selected from O, S and N, wherein a —CH₂— group can optionally be replaced by a —C(O)— group and wherein a sulphur atom in the ring may optionally be oxidised to a S(O) or S(O)₂ group; which ring is optionally substituted on an available carbon or nitrogen atom by 1 or 2 substituents independently selected from R⁸; or

HET-3 is an 6-10 membered bicyclic saturated or partially unsaturated heterocyclyl ring, optionally containing 1 further nitrogen atom (in addition to the linking N atom) wherein a —CH₂— group can optionally be replaced by a —C(O)—; which ring is optionally substituted on an available carbon or nitrogen atom by 1 substituent selected from hydroxy (not on nitrogen) and R³;

R⁸ is selected from —OR⁵, (1-4C)alkyl, —C(O)(1-4C)alkyl, —C(O)NR⁴R⁵, (1-4C)alkylamino, di(1-4C)alkylamino, HET-3 (wherein said ring is unsubstituted), (1-4C)alkoxy(1-4C)alkyl, hydroxy(1-4C)alkyl and —S(O)pR⁵;

HET-4 is a 5- or 6-membered, C- or N-linked unsubstituted heteroaryl ring containing 1, 2 or 3 ring heteroatoms independently selected from O, N and S;

p is (independently at each occurrence) 0, 1 or 2;

m is 0 or 1;

n is 0, 1 or 2;

provided that when m is 0, then n is 1 or 2;

or a salt, pro-drug or solvate thereof.

It will be understood that when R⁴ is —C(O)NR⁵R⁵, each R⁵ is independently selected from hydrogen and (1-4C)alkyl, and therefore this definition of R⁴ includes (but is not limited to) —CONH₂, —CONHMe, —CONMe₂ and —CONMeEt.

It will be understood that where a compound of the formula (I) contains more than one HET-2 ring, they may be the same or different.

It will be understood that where a compound of the formula (I) contains more than one group R⁴, they may be the same or different.

It will be understood that where a compound of the formula (I) contains more than one group R⁵, they may be the same or different.

It will be understood that where a compound of the formula (I) contains more than one group R⁸, they may be the same or different.

A similar convention applies for all other groups and substituents on a compound of formula (I) as hereinbefore defined.

It will be understood that any single carbon atom in HET-1 may only be substituted by one group R⁶ in order to maintain aromaticity of the ring. Up to two different carbon atoms in a HET-1 ring may be substituted by an R⁶ group, each of which may be the same or different, provided the structure thereby formed is stable and aromatic.

It will be understood that R⁸ can be present on any or all available carbon atoms in the heterocyclic ring (HET-3) formed by NR⁴R⁵; each carbon atom can be substituted with 1 or 2 R⁸ groups which may be the same or different, provided the structure thereby formed is stable (so, for example, it is not intended to cover gem-dihydroxy substitution). Similarly any available nitrogen atom may be substituted by R⁸ provided substitution does not lead to quaternisation of the nitrogen. Preferably, the heterocyclic ring (HET-3) formed by NR⁴R⁵ is mono-substituted on one nitrogen or carbon atom, or is unsubstituted.

Compounds of Formula (I) may form salts which are within the ambit of the invention. Pharmaceutically acceptable salts are preferred although other salts may be useful in, for example, isolating or purifying compounds.

In another aspect, the invention relates to compounds of formula (I) as hereinabove defined or to a pharmaceutically acceptable salt.

In another aspect, the invention relates to compounds of formula (I) as hereinabove defined or to a pro-drug thereof. Suitable examples of pro-drugs of compounds of formula (I) are in-vivo hydrolysable esters of compounds of formula (I). Therefore in another aspect, the invention relates to compounds of formula (I) as hereinabove defined or to an in-vivo hydrolysable ester thereof.

In this specification the generic term “alkyl” includes both straight-chain and branched-chain alkyl groups. However references to individual alkyl groups such as “propyl” are specific for the straight chain version only and references to individual branched-chain alkyl groups such as t-butyl are specific for the branched chain version only. For example,

“(1-4C)alkyl” includes methyl, ethyl, propyl, isopropyl and t-butyl. An analogous convention applies to other generic terms.

For the avoidance of doubt, reference to the group HET-1 containing a nitrogen in the 2-position, is intended to refer to the 2-position relative to the amide nitrogen atom to which the group is attached. For example, the following structures are encompassed (but not limiting on the invention):

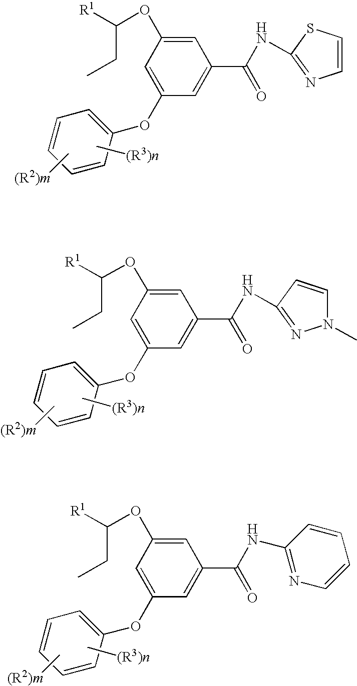

Suitable examples of HET-1 as a 5- or 6-membered, C-linked heteroaryl ring as hereinbefore defined, include thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl and triazolyl.

It will be understood that HET-2 can be a saturated, or partially or fully unsaturated ring.

Suitable examples of HET-2 include azetidinyl, furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, morpholino, morpholinyl, piperidinyl, piperazinyl, morpholinyl, thiomorpholinyl, pyrrolyl, pyrrolidinyl, pyrrolidonyl, 2,5-dioxopyrrolidinyl, 1,1-dioxotetrahydrothienyl, 2-oxoimidazolidinyl, 2,4-dioxoimidazolidinyl, 2-oxo-1,3,4-(4-triazolinyl), 2-oxazolidinonyl, 2-oxotetrahydrofuranyl, tetrahydrofuranyl, tetrahydropyranyl, 1,1-dioxothiomorpholino, 1,3-dioxolanyl, 1,2,4-triazolyl, 1,2,3-triazolyl, pyranyl, and 4-pyridonyl.

It will be understood that HET-2 may be linked by any appropriate available C or N atom, therefore for example, for HET-2 as “imidazolyl” includes 1-, 2-, 4- and 5-imidazolyl.

Suitable examples of HET-3 as a 4-6 membered saturated or partially unsaturated heterocyclic ring are morpholino, piperidinyl, piperazinyl, pyrrolidinyl and azetidinyl.

A suitable example of HET-3 as a 7-membered saturated or partially unsaturated heterocyclic ring is homopiperazinyl, homo-morpholino, homo-thiomorpholino (and versions thereof wherein the sulfur is oxidised to an SO or S(O)₂ group) and homo-piperidinyl.

Suitable examples of HET-3 as an 6-10 membered bicyclic heterocyclic ring are bicyclic saturated or partially unsaturated heterocyclyl ring such as those illustrated by the structures shown below (wherein the dotted line indicates the point of attachment to the rest of the molecule):

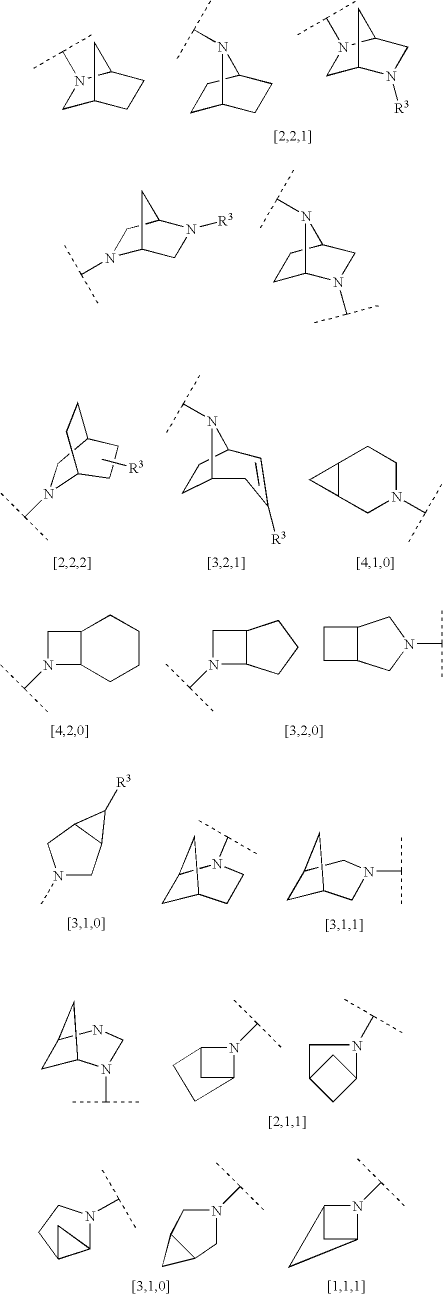

In particular HET-3 is a [2,2,1] system such as

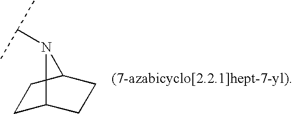

In another embodiment, HET-3 is a [2.1.1] system such as

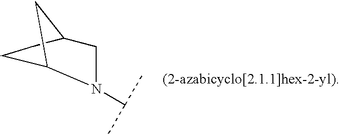

Suitable examples of HET-4 are furyl, pyrrolyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl and triazolyl.

It will be appreciated that, where definitions of heterocyclyl groups HET-1 to HET-4 encompass heteroaryl or heterocyclyl rings which may be substituted on nitrogen, such substitution may not result in charged quaternary nitrogen atoms or unstable structures (such as N-halo compounds). It will be appreciated that the definitions of HET-1 to HET-4 are not intended to include any O—O, O—S or S—S bonds. It will be appreciated that the definitions of HET-1 to HET-4 are not intended to include unstable structures.

Examples of (1-4C)alkyl include methyl, ethyl, propyl, isopropyl, butyl and tert-butyl; examples of (3-6C)cycloalkyl include cyclopropyl, cyclobutyl, cyclopentyl and cyclohexyl; examples of halo include fluoro, chloro, bromo and iodo; examples of hydroxy(1-4C)alkyl include hydroxymethyl, 1-hydroxyethyl, 2-hydroxyethyl, 2-hydroxypropyl, 3-hydroxypropyl, 1-hydroxyisopropyl and 4-hydroxybutyl; examples of (1-4C)alkoxy(1-4C)alkyl include methoxymethyl, ethoxymethyl, tert-butoxymethyl, 2-methoxyethyl, 2-ethoxyethyl, methoxypropyl, 2-methoxypropyl and methoxybutyl; examples of (1-4C)alkylS(O)p(1-4C)alkyl include methylsulfinylmethyl, ethylsulfinylmethyl, ethylsulfinylethyl, methylsulfinylpropyl, methylsulfinylbutyl, methylsulfonylmethyl, ethylsulfonylmethyl, ethylsulfonylethyl, methylsulfonylpropyl, methylsulfonylbutyl, methylthiomethyl, ethylthiomethyl, ethylthioethyl, methylthiopropyl, and methylthiobutyl; examples of amino(1-4C)alkyl include aminomethyl, aminoethyl, 2-aminopropyl, 3-aminopropyl, 1-aminoisopropyl and 4-aminobutyl; examples of (1-4C)alkylamino(1-4C)alkyl include (N-methyl)aminomethyl, (N-ethyl)aminomethyl, 1-((N-methyl)amino)ethyl, 2-((N-methyl)amino)ethyl, (N-ethyl)aminoethyl, (N-methyl)aminopropyl, and 4-((N-methyl)amino)butyl; examples of di(1-4C)alkylamino(1-4C)alkyl include dimethylaminomethyl, methyl(ethyl)aminomethyl, methyl(ethyl)aminoethyl, (N,N-diethyl)aminoethyl, (N,N-dimethyl)aminopropyl and (N,N-dimethyl)aminobutyl; examples of (1-4C)alkylamino include methylamino, ethylamino, propylamino, isopropylamino, butylamino and tert-butylamino; examples of di(1-4C)alkylamino include dimethylamino, methyl(ethyl)amino, diethylamino, dipropylamino, di-isopropylamino and dibutylamino; examples of —C(O)(1-4C)alkyl include methylcarbonyl, ethylcarbonyl, propylcarbonyl and tert-butyl carbonyl.

It is to be understood that, insofar as certain of the compounds of Formula (I) defined above may exist in optically active or racemic forms by virtue of one or more asymmetric carbon atoms, the invention includes in its definition any such optically active or racemic form which possesses the property of stimulating GLK directly or inhibiting the GLK/GLKRP interaction. The synthesis of optically active forms may be carried out by standard techniques of organic chemistry well known in the art, for example by synthesis from optically active starting materials or by resolution of a racemic form. It is also to be understood that certain compounds may exist in tautomeric forms and that the invention also relates to any and all tautomeric forms of the compounds of the invention which activate GLK.

It is also to be understood that certain compounds of the formula (I) and salts thereof can exist in solvated as well as unsolvated forms such as, for example, hydrated forms. It is to be understood that the invention encompasses all such solvated forms which activate GLK.

In one embodiment of the invention are provided compounds of formula (I), in an alternative embodiment are provided pharmaceutically-acceptable salts of compounds of formula (I), in a further alternative embodiment are provided in-vivo hydrolysable esters of compounds of formula (I), and in a further alternative embodiment are provided pharmaceutically-acceptable salts of in-vivo hydrolysable esters of compounds of formula (I).

Preferred values of each variable group are as follows. Such values may be used where appropriate with any of the values, definitions, claims, aspects or embodiments defined hereinbefore or hereinafter. In particular, each may be used as an individual limitation on the broadest definition of formula (I). Further, each of the following values may be used in combination with one or more of the other following values to limit the broadest definition of formula (I).
(1) R¹ is hydroxymethyl and the configuration is preferably (S), that is: 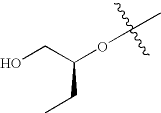
(2) R² is —C(O)NR⁴R⁵
(3) R² is —SO₂NR⁴R⁵
(4) R² is —S(O)ₚR⁴
(5) R² is HET-2
(6) m is 1 and R² is in the para position relative to the ether linkage
(7) m is 1 and n is 0 or 1
(8) m is 1 and n is 0
(9) m is 1, n is 0 and R² is in the para position relative to the ether linkage
(10) m is 1, n is 1, R² is in the para position relative to the ether linkage, R³ is in the ortho position relative to the ether linkage
(11) m is 1, n is 1, R² is in the para position relative to the ether linkage, R³ is in the ortho position relative to the ether linkage
(12) m is 1, n is 1, R² is in the para position relative to the ether linkage, R³ is in the meta position relative to the ether linkage
(13) n is 0
(14) n is 1
(15) n is 2
(16) n is 2 and both R³ are halo
(17) n is 2 and each R³ is independently halo or methoxy
(18) m is 1, n is 2 and R² is in the para position relative to the ether linkage
(19) m is 1, n is 2, R² is in the para position relative to the ether linkage and each R³ is in an ortho position relative to the ether linkage
(20) m is 1, n is 2, both R³ are halo, R² is in the para position relative to the ether linkage and each R³ is in an ortho position relative to the ether linkage
(21) m is 1, n is 2, both R³ are halo, R² is in the para position relative to the ether linkage and one R³ is in an ortho position relative to the ether linkage and the other R³ is in a meta position relative to the ether linkage
(22) R³ is fluoromethyl or difluoromethyl
(23) R³ is halo or trifluoromethyl
(24) R³ is halo
(25) R³ is chloro or fluoro
(26) R³ is fluoro
(27) R³ is methoxy
(28) n is 2 and both R³ are fluoro
(29) n is 2 and one R³ is fluoro and the other is chloro
(30) n is 2, both R³ are fluoro and are in the 3- and 5-positions (meta-positions) relative to the ether linkage
(31) m is 1, n is 2, R² is in the para position relative to the ether linkage, both R³ are fluoro and are in the 3- and 5-positions relative to the ether linkage
(32) p is 0
(33) p is 1
(34) p is 2
(35) HET-1 is a 5-membered heteroaryl ring
(36) HET-1 is a 6-membered heteroaryl ring
(37) HET-1 is substituted with 1 or 2 substituents independently selected from R⁶
(38) HET-1 is substituted with 1 substituent selected from R⁶
(39) HET-1 is unsubstituted
(40) HET-1 is selected from thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, and triazolyl
(41) HET-1 is selected from thiazolyl, isothiazolyl, thiadiazolyl, pyrazolyl, imidazolyl, oxazolyl, isoxazolyl and oxadiazolyl
(42) HET-1 is selected from pyridyl, pyrazinyl, pyridazinyl and pyrimidinyl
(43) HET-1 is selected from thiazolyl, pyrazolyl and oxazolyl
(44) HET-1 is selected from thiadiazolyl and oxadiazolyl
(45) HET-1 is selected from 1,3,4-thiadiazolyl and 1,3,4-oxadiazolyl
(46) HET-1 is selected from 1,2,4-oxadiazolyl and 1,2,4-oxadiazolyl
(47) HET-1 is pyrazolyl, particularly N-methyl or N-ethylpyrazolyl
(48) HET-1 is pyridyl or pyrazinyl
(49) HET-1 is pyrazinyl
(50) HET-1 is selected from thiazolyl, pyrazolyl, thiadiazolyl and pyrazinyl;
(51) R⁶ is selected from (1-4C)alkyl, halo, hydroxy(1-4C)alkyl, di(1-4C)alkylamino(1-4C)alkyl and HET-4
(52) R⁶ is selected from methyl, ethyl, bromo, chloro, fluoro, hydroxymethyl, methoxymethyl,
aminomethyl, N-methylaminomethyl, dimethylaminomethyl
(53) R⁶ is selected from (1-4C)alkyl, halo, hydroxy(1-4C)alkyl, (1-4C)alkoxy(1-4C)alkyl, (1-4C)alkylS(O)p(1-4C)alkyl, amino(1-4C)alkyl, (1-4C)alkylamino(1-4C)alkyl, and di(1-4C)alkylamino(1-4C)alkyl
(54) R⁶ is selected from methyl, ethyl, bromo, chloro, fluoro, aminomethyl, N-methylaminomethyl, and dimethylaminomethyl
(55) R⁶ is selected from methyl, ethyl, bromo, chloro, fluoro, hydroxymethyl and methoxymethyl
(56) R⁶ is selected from methyl, ethyl, bromo, chloro and fluoro
(49) R⁶ is methyl
(57) R⁶ is selected from methyl, ethyl, bromo, chloro, fluoro, aminomethyl, N-methylaminomethyl, dimethylaminomethyl, hydroxymethyl and methoxymethyl
(58) R⁶ is selected from methyl, ethyl, aminomethyl, N-methylaminomethyl, dimethylaminomethyl, hydroxymethyl and methoxymethyl
(59) R⁶ is selected from (1-4C)alkyl and (1-4C)alkoxy(1-4C)alkyl
(60) R⁶ is selected from methyl, ethyl, isopropyl and methoxymethyl
(61) when 2 substituents R⁶ are present, both are selected from methyl, ethyl, bromo, chloro and fluoro; preferably both are methyl
(62) R⁶ is selected from (1-4C)alkylS(O)p(1-4C)alkyl, (1-4C)alkylamino(1-4C)alkyl, di(1-4C)alkylamino(1-4C)alkyl and HET-4
(63) R⁶ is HET-4
(64) HET-4 is selected from furyl, pyrrolyl and thienyl
(65) HET-4 is furyl
(66) R⁴ is hydrogen
(67) R⁴ is (1-4C)alkyl [substituted by 1 or 2 substituents independently selected from HET-2, —OR⁵, —SO₂R⁵, (3-6C)cycloalkyl (optionally substituted with 1 group selected from R⁷) and —C(O)NR⁵R⁵]
(68) R⁴ is (1-4C)alkyl [substituted by 1 substituent selected from HET-2, —OR⁵, —SO₂R⁵, (3-6C)cycloalkyl and —C(O)NR⁵R⁵]
(69) R⁴ is (1-4C)alkyl
(70) R⁴ is (1-4C)alkyl substituted by —OR⁵
(71) R⁴ is (1-4C)alkyl substituted by HET-2
(72) R⁴ is (3-6C)cycloalkyl, particularly cyclopropyl or cyclobutyl
(73) R⁴ is (3-6C)cycloalkyl substituted by a group selected from R⁷
(74) R⁴ is (3-6C)cycloalkyl substituted by a group selected from —OR⁵ and (1-4C)alkyl
(75) R⁴ is selected from (1-4C)alkyl and (3-6C)cycloalkyl
(76) R⁴ is selected from methyl, ethyl, cyclopropyl and cyclobutyl
(77) R⁴ is HET-2
(78) R⁴ is selected from hydrogen, (1-4C)alkyl, and (1-4C)alkyl substituted with —OR⁵
(79) HET-2 is unsubstituted
(80) HET-2 is substituted with 1 or 2 substituents independently selected from (1-4C)alkyl, hydroxy and (1-4C)alkoxy
(81) HET-2 is a fully saturated ring system
(82) HET-2 is a fully unsaturated ring system
(83) HET-2 is selected from azetidinyl, morpholino, morpholinyl, piperidinyl, piperazinyl, 3-oxopiperazinyl, thiomorpholinyl, pyrrolidinyl, pyrrolidonyl, 2,5-dioxopyrrolidinyl, 1,1-dioxotetrahydrothienyl, 2-oxazolidinonyl, 2-oxotetrahydrofuranyl, tetrahydrofuranyl, tetrahydropyranyl, 1,1-dioxothiomorpholino, 1,3-dioxolanyl, 2-oxoimidazolidinyl, 2,4-dioxoimidazolidinyl, pyranyl and 4-pyridonyl
(84) HET-2 is selected from azetidinyl, morpholino, morpholinyl, piperidinyl, piperazinyl, pyrrolidinyl, thiomorpholinyl, tetrahydrofuranyl, and tetrahydropyranyl
(85) HET-2 is selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, pyrrolyl, 1,2,4-triazolyl and 1,2,3-triazolyl
(86) HET-2 is selected from furyl, thienyl, thiazolyl, isothiazolyl, thiadiazolyl, pyridyl, imidazolyl, pyrimidinyl, oxazolyl, isoxazolyl, oxadiazolyl, piperidinyl, piperazinyl, 3-oxopiperazinyl, pyrrolidinyl, pyrrolidonyl, 2-oxazolidinonyl, tetrahydrofuranyl, tetrahydropyranyl, 1,1-dioxotetrahydrothienyl, and 2-oxoimidazolidinyl
(87) HET-2 is selected from morpholino, furyl, imidazolyl, oxazolyl, isoxazolyl, oxadiazolyl, piperidinyl, piperazinyl, 3-oxopiperazinyl, pyrrolidinyl, 2-pyrrolidonyl, 2-oxazolidinonyl, tetrahydrofuranyl, tetrahydropyranyl, 1,1-dioxotetrahydrothienyl, and 2-oxoimidazolidinyl
(88) HET-2 is selected from morpholino, furyl, imidazolyl, isoxazolyl, oxadiazolyl, piperidinyl, piperazinyl, 3-oxopiperazinyl, pyrrolidinyl, 2-pyrrolidonyl, tetrahydropyranyl, 1,1-dioxotetrahydrothienyl, and 2-oxoimidazolidinyl
(89) HET-2 is oxadiazolyl or pyrazolyl
(90) R⁵ is hydrogen
(91) R⁵ is (1-4)alkyl, preferably methyl
(92) R⁵ is hydrogen or methyl
(93) R⁷ is selected from —OR⁵, (1-4C)alkyl, —C(O)(1-4C)alkyl, —C(O)NR⁴R⁵, (1-4C)alkoxy(1-4C)alkyl, and hydroxy(1-4C)alkyl
(94) R⁷ is selected from —OR⁵, (1-4C)alkyl, —C(O)(1-4C)alkyl, —C(O)NR⁴R⁵, and hydroxy(1-4C)alkyl
(95) R⁷ is selected from hydroxy, methoxy, —COMe, —CONH₂, —CONHMe, —CONMe₂, and hydroxymethyl
(96) R⁷ is selected from (1-4C)alkyl, hydroxy and (1-4C)alkoxy
(97) R⁷ is selected from methyl, ethyl, methoxy and hydroxy
(98) R⁸ is selected from methyl, hydroxy, methoxy, —COMe, —CONH₂, —CONHMe, —CONMe₂, hydroxymethyl, hydroxyethyl, —NHMe and —NMe₂(99) R⁸ is selected from morpholino, piperidinyl, piperazinyl, pyrrolidinyl and azetidinyl
(100) R⁸ is selected from methyl, —COMe, —CONH₂, hydroxyethyl and hydroxy
(101) R⁸ is selected from (1-4C)alkyl and (1-4C)alkoxy
(102) R⁸ is selected from methyl, methoxy and isopropoxy
(103) HET-3 is a fully saturated ring
(104) HET-3 is selected from morpholino, piperidinyl, piperazinyl, pyrrolidinyl and azetidinyl
(105) R⁴ and R⁵ together with the nitrogen to which they are attached form a ring as defined by HET-3
(106) HET-3 is selected from pyrrolidinyl and azetidinyl
(107) HET-3 is azetidinyl
(108) HET-3 is a 4 to 6-membered saturated or partially unsaturated heterocyclic ring as hereinbefore defined
(109) HET-3 is a 7-membered saturated or partially unsaturated heterocyclic ring as hereinbefore defined
(110) HET-3 is an 6 to 10-membered bicyclic saturated or partially unsaturated heterocyclic ring as hereinbefore defined
(111) HET-3 is 7-azabicyclo[2.2.1]hept-7-yl
(112) HET-3 is 7-azabicyclo[2.2.1]hept-7-yl or 2-azabicyclo[2.1.1]hex-2-yl
(113) HET-3 is selected from morpholino, piperidinyl, piperazinyl, pyrrolidinyl and azetidinyl
(114) HET-3 is unsubstituted
(115) HET-3 is substituted by methyl, methoxy or isopropoxy

According to a further feature of the invention there is provided the following preferred groups of compounds of the invention:

In a further aspect of the invention there is provided a compound of Formula (I) wherein:

R¹ is hydroxymethyl;

R² is selected from —C(O)NR⁴R⁵, —SO₂NR⁴R⁵, —S(O)ₚR⁴ and HET-2;

HET-1 is a 5- or 6-membered, C-linked heteroaryl ring containing a nitrogen atom in the 2-position and optionally 1, 2 or 3 further ring heteroatoms independently selected from O, N and S; which ring is optionally substituted on an available carbon atom, or on a ring nitrogen atom provided it is not thereby quaternised, with 1 or 2 substituents independently selected from R⁶;

HET-2 is a 5- or 6-membered, C- or N-linked heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to an S(O) or S(O)₂ group, which ring is optionally substituted on an available carbon or nitrogen atom by 1 or 2 substituents independently selected from R⁷;

R³ is selected from halo, fluoromethyl, difluoromethyl, trifluoromethyl, methyl, methoxy and cyano;

R⁴ is selected from hydrogen, (1-4C)alkyl [optionally substituted by —OR⁵], (3-6C)cycloalkyl [optionally substituted by —OR⁵], (3-6C)cycloalkyl (optionally substituted with 1 group selected from R⁷) and HET-2;

R⁵ is hydrogen or (1-4C)alkyl;

or R⁴ and R⁵ together with the nitrogen atom to which they are attached may form a 4-6 membered heterocyclyl ring system as defined by HET-3;

R⁶ is independently selected from (1-4C)alkyl, halo, hydroxy(1-4C)alkyl, (1-4C)alkoxy(1-4C)alkyl, (1-4C)alkylS(O)p(1-4C)alkyl, amino(1-4C)alkyl, (1-4C)alkylamino(1-4C)alkyl, di(1-4C)alkylamino(1-4C)alkyl and HET-4;

R⁷ is selected from —OR⁵ and (1-4C)alkyl;

HET-3 is an N-linked, 4 to 6 membered, saturated or partially unsaturated heterocyclyl ring, optionally containing 1 or 2 further heteroatoms (in addition to the linking N atom) independently selected from O, N and S, wherein a —CH₂— group can optionally be replaced by a —C(O)— and wherein a sulphur atom in the ring may optionally be oxidised to an S(O) or S(O)₂ group; which ring is optionally substituted on an available carbon or nitrogen atom by 1 or 2 substituents independently selected from R⁸;

R⁸ is selected from —OR⁵ and (1-4C)alkyl;

HET-4 is a 5- or 6-membered, C- or N-linked unsubstituted heteroaryl ring containing 1, 2 or 3 ring heteroatoms independently selected from O, N and S;

p is (independently at each occurrence) 0, 1 or 2;

m is 0 or 1;

n is 0, 1 or 2;

provided that when m is 0, then n is 1 or 2;

or a salt, pro-drug or solvate thereof.

In a further aspect of the invention there is provided a compound of Formula (I) wherein:

R¹ is hydroxymethyl;

R² is selected from —C(O)NR⁴R⁵, —SO₂NR⁴R⁵, —S(O)ₚR⁴ and HET-2;

HET-1 is a 5- or 6-membered, C-linked heteroaryl ring containing a nitrogen atom in the 2-position and optionally 1, 2 or 3 further ring heteroatoms independently selected from O, N and S; which ring is optionally substituted on an available carbon atom, or on a ring nitrogen atom provided it is not thereby quaternised, with 1 or 2 substituents independently selected from R⁶;

HET-2 is a 5- or 6-membered, C- or N-linked heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to an S(O) or S(O)₂ group, which ring is optionally substituted on an available carbon or nitrogen atom by 1 or 2 substituents independently selected from R⁷;

R³ is selected from halo, fluoromethyl, difluoromethyl, trifluoromethyl, methyl, methoxy and cyano;

R⁴ is selected from hydrogen, (1-4C)alkyl [optionally substituted by —OR⁵], (3-6C)cycloalkyl [optionally substituted by —OR⁵], (3-6C)cycloalkyl (optionally substituted with 1 group selected from R⁷) and HET-2;

R⁵ is hydrogen or (1-4C)alkyl;

or R⁴ and R⁵ together with the nitrogen atom to which they are attached may form a heterocyclyl ring system as defined by HET-3;

R⁶ is independently selected from (1-4C)alkyl, halo, hydroxy(1-4C)alkyl, (1-4C)alkoxy(1-4C)alkyl, (1-4C)alkylS(O)p(1-4C)alkyl, amino(1-4C)alkyl, (1-4C)alkylamino(1-4C)alkyl, di(1-4C)alkylamino(1-4C)alkyl and HET-4;

R⁷ is selected from —OR⁵ and (1-4C)alkyl;

HET-3 is an N-linked, 4 to 6 membered, saturated or partially unsaturated heterocyclyl ring, optionally containing 1 or 2 further heteroatoms (in addition to the linking N atom) independently selected from O, N and S, wherein a —CH₂— group can optionally be replaced by a —C(O)— and wherein a sulphur atom in the ring may optionally be oxidised to an S(O) or S(O)₂ group; which ring is optionally substituted on an available carbon or nitrogen atom by 1 or 2 substituents independently selected from R⁸; or

HET-3 is an N-linked, 7 membered, saturated or partially unsaturated heterocyclyl ring, optionally containing 1 further heteroatom (in addition to the linking N atom) independently selected from O, S and N, wherein a —CH₂— group can optionally be replaced by a —C(O)— group and wherein a sulphur atom in the ring may optionally be oxidised to an S(O) or S(O)₂ group; which ring is optionally substituted on an available carbon or nitrogen atom by 1 or 2 substituents independently selected from R⁸; or HET-3 is an 6-10 membered bicyclic saturated or partially unsaturated heterocyclyl ring, optionally containing 1 further nitrogen atom (in addition to the linking N atom), wherein a —CH₂— group can optionally be replaced by a —C(O)—; which ring is optionally substituted on an available carbon or nitrogen atom by 1 substituent selected from hydroxy (not on nitrogen) and R³;

R⁸ is selected from —OR⁵ and (1-4C)alkyl;

HET-4 is a 5- or 6-membered, C- or N-linked unsubstituted heteroaryl ring containing 1, 2 or 3 ring heteroatoms independently selected from O, N and S;

p is (independently at each occurrence) 0, 1 or 2;

m is 0 or 1;

n is 0, 1 or 2;

provided that when m is 0, then n is 1 or 2;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is a 5- or 6-membered heteroaryl ring as hereinbefore defined;

R² is —CONR⁴R⁵ or —SO₂NR⁴R⁵;

R³ is halo or trifluoromethyl;

R⁴ is (1-4C)alkyl [optionally substituted by 1 or 2 substituents independently selected from HET-2, —OR⁵, —SO₂R⁵, (3-6C)cycloalkyl (optionally substituted with 1 group selected from R⁷) and —C(O)NR⁵R⁵];

R⁵ is hydrogen or methyl;

HET-2 is a 5- or 6-membered heterocyclyl ring as hereinbefore defined, containing 1 or 2 heteroatoms independently selected from O, N and S; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is a 5- or 6-membered heteroaryl ring and is optionally substituted by a group R⁶;

R² is —CONR⁴R⁵ or —SO₂NR⁴R⁵;

R³ is halo or trifluoromethyl;

R⁴ is (1-4C)alkyl [optionally substituted by 1 or 2 substituents independently selected from HET-2, —OR⁵, —SO₂R⁵, (3-6C)cycloalkyl (optionally substituted with 1 group selected from R⁷) and —C(O)NR⁵R⁵];

R⁵ is hydrogen or methyl;

R⁶ is selected from methyl, ethyl, bromo, chloro, fluoro, hydroxymethyl, methoxymethyl, aminomethyl, N-methylaminomethyl, and dimethylaminomethyl;

HET-2 is an optionally substituted 5- or 6-membered heterocyclyl ring as hereinbefore defined, containing 1 or 2 heteroatoms independently selected from O, N and S; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from thiazolyl, isothiazolyl, thiadiazolyl, pyrazolyl, imidazolyl, oxazolyl, isoxazolyl and oxadiazolyl and is optionally substituted by a group R⁶;

R² is —CONR⁴R⁵ or —SO₂NR⁴R⁵;

R³ is halo or trifluoromethyl;

R⁴ is (1-4C)alkyl [optionally substituted by 1 or 2 substituents independently selected from HET-2, —OR⁵, —SO₂R⁵, (3-6C)cycloalkyl and —C(O)NR⁵R⁵];

R⁵ is hydrogen or methyl;

R⁶ is selected from methyl, ethyl, bromo, chloro, fluoro, hydroxymethyl, methoxymethyl, aminomethyl, N-methylaminomethyl, and dimethylaminomethyl;

HET-2 is selected from azetidinyl, morpholino, morpholinyl, piperidinyl, piperazinyl, 3-oxopiperazinyl, thiomorpholinyl, pyrrolidinyl, pyrrolidonyl, 2,5-dioxopyrrolidinyl, 1,1-dioxotetrahydrothienyl, 2-oxazolidinonyl, 2-oxotetrahydrofuranyl, tetrahydrofuranyl, tetrahydropyranyl, 1,1-dioxothiomorpholino, 1,3-dioxolanyl, 2-oxoimidazolidinyl, 2,4-dioxoimidazolidinyl, pyranyl and 4-pyridonyl; wherein HET-2 is optionally substituted by R⁷; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from pyridyl, pyrazinyl, pyridazinyl and pyrimidinyl and is optionally substituted by a group R⁶;

R² is —CONR⁴R⁵ or —SO₂NR⁴R⁵;

R³ is halo or trifluoromethyl;

R⁴ is (1-4C)alkyl [optionally substituted by 1 or 2 substituents independently selected from HET-2, —OR⁵, —SO₂R⁵, (3-6C)cycloalkyl and —C(O)NR⁵R⁵];

R⁵ is hydrogen or methyl;

R⁶ is selected from methyl, ethyl, bromo, chloro, fluoro, hydroxymethyl, methoxymethyl, aminomethyl, N-methylaminomethyl, and dimethylaminomethyl;

HET-2 is selected from azetidinyl, morpholino, morpholinyl, piperidinyl, piperazinyl, 3-oxopiperazinyl, thiomorpholinyl, pyrrolidinyl, pyrrolidonyl, 2,5-dioxopyrrolidinyl, 1,1-dioxotetrahydrothienyl, 2-oxazolidinonyl, 2-oxotetrahydrofuranyl, tetrahydrofuranyl, tetrahydropyranyl, 1,1-dioxothiomorpholino, 1,3-dioxolanyl, 2-oxoimidazolidinyl, 2,4-dioxoimidazolidinyl, pyranyl and 4-pyridonyl; wherein HET-2 is optionally substituted by R⁷; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from thiazolyl, isothiazolyl, thiadiazolyl, pyrazolyl, imidazolyl, oxazolyl, isoxazolyl and oxadiazolyl and is optionally substituted by a group R⁶;

R² is —CONR⁴R⁵ or —SO₂NR⁴R⁵;

R³ is halo or trifluoromethyl;

R⁴ is (1-4C)alkyl [optionally substituted by 1 or 2 substituents independently selected from HET-2, —OR⁵, —SO₂R⁵, (3-6C)cycloalkyl and —C(O)NR⁵R⁵];

R⁵ is hydrogen or methyl;

R⁶ is selected from methyl, ethyl, bromo, chloro, fluoro, hydroxymethyl, methoxymethyl, aminomethyl, N-methylaminomethyl, and dimethylaminomethyl;

HET-2 is selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, pyrrolyl, 1,2,4-triazolyl and 1,2,3-triazolyl; wherein HET-2 is optionally substituted by R⁷; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from pyridyl, pyrazinyl, pyridazinyl and pyrimidinyl and is optionally substituted by a group R⁶;

R² is —CONR⁴R⁵ or —SO₂NR⁴R⁵;

R³ is halo or trifluoromethyl;

R⁴ is (1-4C)alkyl [optionally substituted by 1 or 2 substituents independently selected from HET-2, —OR⁵, —SO₂R⁵, (3-6C)cycloalkyl and —C(O)NR⁵R⁵];

R⁵ is hydrogen or methyl;

R⁶ is selected from methyl, ethyl, bromo, chloro, fluoro, hydroxymethyl, methoxymethyl, aminomethyl, N-methylaminomethyl, and dimethylaminomethyl;

HET-2 is selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, pyrrolyl, 1,2,4-triazolyl and 1,2,3-triazolyl; wherein HET-2 is optionally substituted by R⁷; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from thiazolyl, isothiazolyl, thiadiazolyl, pyrazolyl, oxazolyl, isoxazolyl and oxadiazolyl and is optionally substituted by a group R⁶;

R² is —CONR⁴R⁵ or —SO₂NR⁴R⁵;

R³ is halo or trifluoromethyl;

R⁴ is selected from hydrogen, (1-4C)alkyl, [optionally substituted by —OR⁵], (3-6C)cycloalkyl (optionally substituted with 1 group selected from R⁷) and HET-2;

R⁵ is hydrogen or methyl;

R⁶ is selected from methyl, ethyl, bromo, chloro, fluoro, hydroxymethyl, methoxymethyl, aminomethyl, N-methylaminomethyl, and dimethylaminomethyl;

HET-2 is selected from morpholino, furyl, imidazolyl, isoxazolyl, oxadiazolyl, piperidinyl, piperazinyl, 3-oxopiperazinyl, pyrrolidinyl, 2-pyrrolidonyl, tetrahydropyranyl, 1,1-dioxotetrahydrothienyl, and 2-oxoimidazolidinyl; wherein HET-2 is optionally substituted by R⁷; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from pyridyl and pyridazinyl and is optionally substituted by a group R⁶;

R² is —CONR⁴R⁵ or —SO₂NR⁴R⁵;

R³ is halo or trifluoromethyl;

R⁴ is selected from hydrogen, (1-4C)alkyl, [optionally substituted by —OR⁵], (3-6C)cycloalkyl (optionally substituted with 1 group selected from R⁷) and HET-2;

R⁵ is hydrogen or methyl;

R⁶ is selected from methyl, ethyl, bromo, chloro, fluoro, hydroxymethyl, methoxymethyl, aminomethyl, N-methylaminomethyl, and dimethylaminomethyl; HET-2 is selected from morpholino, furyl, imidazolyl, isoxazolyl, oxadiazolyl, piperidinyl, piperazinyl, 3-oxopiperazinyl, pyrrolidinyl, 2-pyrrolidonyl, tetrahydropyranyl, 1,1-dioxotetrahydrothienyl, and 2-oxoimidazolidinyl; wherein HET-2 is optionally substituted by R⁷; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from thiazolyl, isothiazolyl, thiadiazolyl, pyrazolyl, oxazolyl, isoxazolyl and oxadiazolyl and is optionally substituted by a group R⁶;

R² is —CONR⁴R⁵ or —SO₂NR⁴R⁵;

R³ is halo or trifluoromethyl;

R⁴ is selected from (1-4C)alkyl, [optionally substituted by —OR⁵], (3-6C)cycloalkyl (optionally substituted with 1 group selected from R⁷) and HET-2;

R⁵ is hydrogen or methyl;

R⁶ is selected from methyl, ethyl, bromo, chloro, fluoro, hydroxymethyl, methoxymethyl, aminomethyl, N-methylaminomethyl, and dimethylaminomethyl;

HET-2 is selected from piperidinyl, piperazinyl, 3-oxopiperazinyl, 2-pyrrolidonyl, 2,5-dioxopyrrolidinyl, 2-oxotetrahydrofuranyl, tetrahydrofuranyl, tetrahydropyranyl, 2-oxoimidazolidinyl, and 2,4-dioxoimidazolidinyl; wherein HET-2 is optionally substituted by R⁷; and

R⁷ is (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from thiazolyl, isothiazolyl, thiadiazolyl, pyrazolyl, oxazolyl, isoxazolyl and oxadiazolyl and is optionally substituted by a group R⁶;

R² is —CONR⁴R⁵ or —SO₂NR⁴R⁵;

R³ is halo or trifluoromethyl;

R⁴ is selected from (1-4C)alkyl, [substituted by —OR⁵], (3-6C)cycloalkyl (optionally substituted with 1 group selected from R⁷) and HET-2;

R⁵ is hydrogen or methyl;

R⁶ is selected from methyl, ethyl, bromo, chloro, fluoro, hydroxymethyl, methoxymethyl, aminomethyl, N-methylaminomethyl, and dimethylaminomethyl;

HET-2 is piperidinyl or piperazinyl; wherein HET-2 is optionally substituted by R⁷; and

R⁷ is (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0;

HET-1 is selected from thiazolyl, thiadiazolyl and pyrazolyl and is optionally substituted by a group R⁶;

R² is —CONR⁴R⁵;

R⁴ is piperidinyl, optionally substituted with methyl;

R⁵ is hydrogen or methyl;

R⁶ is methyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from pyridyl and pyridazinyl and is optionally substituted by a group R⁶;

R² is —CONR⁴R⁵ or —SO₂NR⁴R⁵;

R³ is halo or trifluoromethyl;

R⁴ is selected from (1-4C)alkyl, [optionally substituted by —OR⁵] and HET-2;

R⁵ is hydrogen or methyl;

R⁶ is selected from methyl, ethyl, bromo, chloro, fluoro, hydroxymethyl, methoxymethyl, aminomethyl, N-methylaminomethyl, and dimethylaminomethyl;

HET-2 is selected from piperidinyl, piperazinyl, 3-oxopiperazinyl, 2-pyrrolidonyl, 2,5-dioxopyrrolidinyl, 2-oxazolidinonyl, 2-oxotetrahydrofuranyl, tetrahydrofuranyl, tetrahydropyranyl, 2-oxoimidazolidinyl, and 2,4-dioxoimidazolidinyl; wherein HET-2 is optionally substituted by R⁷; and

R⁷ is (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from pyridyl and pyridazinyl and is optionally substituted by a group R⁶;

R² is —CONR⁴R⁵ or —SO₂NR⁴R⁵;

R³ is halo or trifluoromethyl;

R⁴ is selected from (1-4C)alkyl, [substituted by —OR⁵] and HET-2;

R⁵ is hydrogen or methyl;

R⁶ is selected from methyl, ethyl, bromo, chloro, fluoro, hydroxymethyl, methoxymethyl, aminomethyl, N-methylaminomethyl, and dimethylaminomethyl;

HET-2 is piperidinyl or piperazinyl; wherein HET-2 is optionally substituted by R⁷; and

R⁷ is (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from thiazolyl, isothiazolyl, thiadiazolyl, pyrazolyl, oxazolyl, isoxazolyl and oxadiazolyl and is optionally substituted by a group R⁶;

R² is —CONR⁴R⁵ or —SO₂NR⁴R⁵;

R³ is halo or trifluoromethyl;

R⁴ and R⁵ together with the nitrogen to which they are attached form a morpholino, piperidinyl, piperazinyl, pyrrolidinyl or azetidinyl ring, which ring is optionally substituted on a carbon or nitrogen atom by R⁸;

R⁶ is selected from methyl, ethyl, bromo, chloro, fluoro, hydroxymethyl, methoxymethyl, aminomethyl, N-methylaminomethyl, and dimethylaminomethyl;

R⁸ is selected from hydroxy, (1-4C)alkoxy and (1-4C)alkyl

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from thiazolyl, isothiazolyl, thiadiazolyl, pyrazolyl, oxazolyl, isoxazolyl and oxadiazolyl and is optionally substituted by a group R⁶;

R² is —CONR⁴R⁵ or —SO₂NR⁴R⁵;

R³ is halo or trifluoromethyl;

R⁴ and R⁵ together with the nitrogen to which they are attached form a morpholino, piperidinyl, piperazinyl, pyrrolidinyl or azetidinyl ring, which ring is optionally substituted on a carbon or nitrogen atom by R⁸;

R⁶ is selected from methyl, ethyl, bromo, chloro, fluoro, hydroxymethyl, methoxymethyl, aminomethyl, N-methylaminomethyl, and dimethylaminomethyl;

R⁸ is pyrrolidine or piperidine;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from thiazolyl, isothiazolyl, thiadiazolyl, pyrazolyl, oxazolyl, isoxazolyl and oxadiazolyl and is optionally substituted by a group R⁶;

R² is —CONR⁴R⁵ or —SO₂NR⁴R⁵;

R³ is halo or trifluoromethyl;

R⁴ and R⁵ together with the nitrogen to which they are attached form a morpholino, piperidinyl, piperazinyl, pyrrolidinyl or azetidinyl ring, which ring is optionally substituted on a carbon or nitrogen atom by (1-4C)alkyl;

R⁶ is selected from methyl, ethyl, bromo, chloro, fluoro, hydroxymethyl, methoxymethyl, aminomethyl, N-methylaminomethyl, and dimethylaminomethyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from pyridyl and pyridazinyl and is optionally substituted by a group R⁶;

R² is —CONR⁴R⁵ or —SO₂NR⁴R⁵;

R³ is halo or trifluoromethyl;

R⁴ and R⁵ together with the nitrogen to which they are attached form a morpholino, piperidinyl, piperazinyl, pyrrolidinyl or azetidinyl ring, which ring is optionally substituted on a carbon or nitrogen atom by (1-4C)alkyl;

R⁶ is selected from methyl, ethyl, bromo, chloro, fluoro, hydroxymethyl, methoxymethyl, aminomethyl, N-methylaminomethyl, and dimethylaminomethyl;

or a salt, pro-drug or solvate thereof.

In a further aspect of the invention is provided a compound of the formula (I) as hereinbefore defined wherein

R¹ is hydroxymethyl;

m is 1 and n is 0;

HET-1 is selected from thiazolyl, thiadiazolyl and pyrazolyl and is optionally substituted by a group R⁶;

R² is —CONR⁴R⁵;

R⁴ and R⁵ together with the nitrogen to which they are attached form a piperidinyl, or piperazinyl ring, which ring is optionally substituted on a carbon or nitrogen atom by (1-4C)alkyl or by a pyrrolidinyl ring;

R⁶ is selected from methyl, ethyl, bromo, chloro, fluoro, hydroxymethyl, methoxymethyl, aminomethyl, N-methylaminomethyl, and dimethylaminomethyl;

or a salt, pro-drug or solvate thereof.

In a further aspect of the invention is provided a compound of the formula (I) as hereinbefore defined wherein

R¹ is hydroxymethyl;

m is 1 and n is 0;

HET-1 is selected from thiazolyl, thiadiazolyl and pyrazolyl and is optionally substituted by a group R⁶;

R² is —CONR⁴R⁵;

R⁴ and R⁵ together with the nitrogen to which they are attached form an azetidinyl ring which ring is optionally substituted on a carbon atom by hydroxy;

R⁶ is selected from methyl, ethyl, bromo, chloro, fluoro, hydroxymethyl, methoxymethyl, aminomethyl, N-methylaminomethyl, and dimethylaminomethyl;

or a salt, pro-drug or solvate thereof.

In a further aspect of the invention is provided a compound of the formula (I) as hereinbefore defined wherein

R¹ is hydroxymethyl;

m is 1 and n is 1;

HET-1 is selected from thiazolyl, thiadiazolyl and pyrazolyl and is optionally substituted by a group R⁶;

R² is —CONR⁴R⁵;

R³ is chloro or fluoro;

R⁴ and R⁵ together with the nitrogen to which they are attached form an azetidinyl ring which ring is optionally substituted on a carbon atom by hydroxy;

R⁶ is selected from methyl, ethyl, bromo, chloro, fluoro, hydroxymethyl, methoxymethyl, aminomethyl, N-methylaminomethyl, and dimethylaminomethyl;

or a salt, pro-drug or solvate thereof.

In a further aspect of the invention is provided a compound of the formula (I) as hereinbefore defined wherein

R¹ is hydroxymethyl;

m is 1 and n is 0;

HET-1 is selected from thiazolyl, thiadiazolyl and pyrazolyl and is optionally substituted by a group R⁶;

R² is —CONR⁴R⁵;

R⁴ and R⁵ together with the nitrogen to which they are attached form a 7-membered ring HET-3 which ring is optionally substituted on a carbon or nitrogen atom by methyl;

R⁶ is selected from methyl, ethyl, bromo, chloro, fluoro, hydroxymethyl, methoxymethyl, aminomethyl, N-methylaminomethyl, and dimethylaminomethyl;

or a salt, pro-drug or solvate thereof.

In a further aspect of the invention is provided a compound of the formula (I) as hereinbefore defined wherein

R¹ is hydroxymethyl;

m is 1 and n is 0;

HET-1 is selected from thiazolyl, thiadiazolyl and pyrazolyl and is optionally substituted by a group R⁶;

R² is —CONR⁴R⁵;

R⁴ and R⁵ together with the nitrogen to which they are attached form an optionally substituted 6-10 membered bicyclic heterocyclic ring HET-3;

R⁶ is selected from methyl, ethyl, bromo, chloro, fluoro, hydroxymethyl, methoxymethyl, aminomethyl, N-methylaminomethyl, and dimethylaminomethyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is a 5- or 6-membered heteroaryl ring;

R¹ is —S(O)pR⁴;

p is 1 or 2;

R³ is halo or trifluoromethyl;

R⁴ is (1-4C)alkyl [optionally substituted by 1 or 2 substituents independently selected from HET-2, —OR⁵, —SO₂R⁵, (3-6C)cycloalkyl (optionally substituted with 1 group selected from R⁷) and —C(O)NR⁵R⁵];

R⁵ is hydrogen or methyl;

HET-2 is a 5- or 6-membered heterocyclyl ring as hereinbefore defined, containing 1 or 2 heteroatoms independently selected from O, N and S; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is a 5- or 6-membered heteroaryl ring and is optionally substituted by a group R⁶;

R¹ is —S(O)pR⁴;

p is 1 or 2;

R³ is halo or trifluoromethyl;

R⁴ is (1-4C)alkyl [optionally substituted by 1 or 2 substituents independently selected from HET-2, —OR⁵, —SO₂R⁵, (3-6C)cycloalkyl (optionally substituted with 1 group selected from R⁷) and —C(O)NR⁵R⁵];

R⁵ is hydrogen or methyl;

R⁶ is selected from methyl, ethyl, bromo, chloro, fluoro, hydroxymethyl, methoxymethyl, aminomethyl, N-methylaminomethyl, and dimethylaminomethyl;

HET-2 is a optionally substituted 5- or 6-membered heterocyclyl ring as hereinbefore defined, containing 1 or 2 heteroatoms independently selected from O, N and S; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from thiazolyl, isothiazolyl, thiadiazolyl, pyrazolyl, imidazolyl, oxazolyl, isoxazolyl and oxadiazolyl and is optionally substituted by a group R⁶;

R¹ is —S(O)pR⁴;

p is 1 or 2;

R³ is halo or trifluoromethyl;

R⁴ is (1-4C)alkyl [optionally substituted by 1 or 2 substituents independently selected from HET-2, —OR⁵, —SO₂R⁵, (3-6C)cycloalkyl and —C(O)NR⁵R⁵];

R⁵ is hydrogen or methyl;

R⁶ is selected from methyl, ethyl, bromo, chloro, fluoro, hydroxymethyl, methoxymethyl, aminomethyl, N-methylaminomethyl, and dimethylaminomethyl;

HET-2 is selected from azetidinyl, morpholino, morpholinyl, piperidinyl, piperazinyl, 3-oxopiperazinyl, thiomorpholinyl, pyrrolidinyl, pyrrolidonyl, 2,5-dioxopyrrolidinyl, 1,1-dioxotetrahydrothienyl, 2-oxazolidinonyl, 2-oxotetrahydrofuranyl, tetrahydrofuranyl, tetrahydropyranyl, 1,1-dioxothiomorpholino, 1,3-dioxolanyl, 2-oxoimidazolidinyl, 2,4-dioxoimidazolidinyl, pyranyl and 4-pyridonyl; wherein HET-2 is optionally substituted by R⁷; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from thiazolyl, isothiazolyl, thiadiazolyl, pyrazolyl, imidazolyl, oxazolyl, isoxazolyl and oxadiazolyl and is optionally substituted by a group R⁶;

R² is —S(O)pR⁴;

p is 1 or 2;

R³ is halo or trifluoromethyl;

R⁴ is selected from hydrogen, (1-4C)alkyl, [optionally substituted by —OR⁵], (3-6C)cycloalkyl (optionally substituted with 1 group selected from R⁷) and HET-2;

R⁵ is hydrogen or methyl;

R⁶ is selected from methyl, ethyl, bromo, chloro, fluoro, hydroxymethyl, methoxymethyl, aminomethyl, N-methylaminomethyl, and dimethylaminomethyl;

HET-2 is selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, pyrrolyl, 1,2,4-triazolyl and 1,2,3-triazolyl; wherein HET-2 is optionally substituted by R⁷; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from pyridyl, pyrazinyl, pyridazinyl and pyrimidinyl and is optionally substituted by a group R⁶;

R¹ is —S(O)pR⁴;

p is 1 or 2;

R³ is halo or trifluoromethyl;

R⁴ is (1-4C)alkyl [optionally substituted by 1 or 2 substituents independently selected from HET-2, —OR⁵, —SO₂R⁵, (3-6C)cycloalkyl and —C(O)NR⁵R⁵];

R⁵ is hydrogen or methyl;

R⁶ is selected from methyl, ethyl, bromo, chloro, fluoro, hydroxymethyl, methoxymethyl, aminomethyl, N-methylaminomethyl, and dimethylaminomethyl;

HET-2 is selected from azetidinyl, morpholino, morpholinyl, piperidinyl, piperazinyl, 3-oxopiperazinyl, thiomorpholinyl, pyrrolidinyl, pyrrolidonyl, 2,5-dioxopyrrolidinyl, 1,1-dioxotetrahydrothienyl, 2-oxazolidinonyl, 2-oxotetrahydrofuranyl, tetrahydrofuranyl, tetrahydropyranyl, 1,1-dioxothiomorpholino, 1,3-dioxolanyl, 2-oxoimidazolidinyl, 2,4-dioxoimidazolidinyl, pyranyl and 4-pyridonyl; wherein HET-2 is optionally substituted by R⁷; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from pyridyl, pyrazinyl, pyridazinyl and pyrimidinyl and is optionally substituted by a group R⁶;

R² is —S(O)pR⁴;

p is 1 or 2;

R³ is halo or trifluoromethyl;

R⁴ is selected from hydrogen, (1-4C)alkyl, [optionally substituted by —OR⁵], (3-6C)cycloalkyl (optionally substituted with 1 group selected from R⁷) and HET-2;

R⁵ is hydrogen or methyl;

R⁶ is selected from methyl, ethyl, bromo, chloro, fluoro, aminomethyl, N-methylaminomethyl, and dimethylaminomethyl;

HET-2 is selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, pyrrolyl, 1,2,4-triazolyl and 1,2,3-triazolyl; wherein HET-2 is optionally substituted by R⁷; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from thiazolyl, isothiazolyl, thiadiazolyl, pyrazolyl, imidazolyl, oxazolyl, isoxazolyl and oxadiazolyl and is optionally substituted by a group R⁶;

R¹ is —S(O)pR⁴;

p is 1 or 2;

R³ is halo or trifluoromethyl;

R⁴ is (1-4C)alkyl;

R⁶ is selected from methyl, ethyl, bromo, chloro, fluoro, hydroxymethyl, methoxymethyl, aminomethyl, N-methylaminomethyl, and dimethylaminomethyl;

or a salt, pro-drug or solvate thereof.

In a further aspect of the invention is provided a compound of the formula (I) as hereinbefore defined wherein

R¹ is hydroxymethyl;

m is 1 and n is 0;

HET-1 is selected from thiazolyl, thiadiazolyl and pyrazolyl and is optionally substituted by a group R⁶;

R¹ is —S(O)pR⁴;

p is 1 or 2;

R⁴ is (1-4C)alkyl;

R⁶ is methyl;

or a salt, pro-drug or solvate thereof.

In a further aspect of the invention is provided a compound of the formula (I) as hereinbefore defined wherein

R¹ is hydroxymethyl;

m is 1 and n is 0;

HET-1 is selected from thiazolyl, thiadiazolyl and pyrazolyl and is optionally substituted by a group R⁶;

R¹ is —S(O)pR⁴;

p is 1 or 2;

R⁴ is (3-6C)cycloalkyl;

R⁶ is methyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from pyridyl, pyrazinyl, pyridazinyl and pyrimidinyl and is optionally substituted by a group R⁶;

R¹ is —S(O)pR⁴;

p is 1 or 2;

R³ is halo or trifluoromethyl;

R⁴ is (1-4C)alkyl;

R⁶ is selected from methyl, ethyl, bromo, chloro, fluoro, hydroxymethyl, methoxymethyl, aminomethyl, N-methylaminomethyl, and dimethylaminomethyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is a 5- or 6-membered heteroaryl ring;

R² is HET-2;

R³ is halo or trifluoromethyl;

R⁵ is hydrogen or (1-4C)alkyl;

HET-2 is a 5- or 6-membered heterocyclyl ring as hereinbefore defined, containing 1 or 2 heteroatoms independently selected from O, N and S; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from thiazolyl, isothiazolyl, thiadiazolyl, pyrazolyl, imidazolyl, oxazolyl, isoxazolyl and oxadiazolyl;

R² is HET-2;

R³ is halo or trifluoromethyl;

R⁵ is hydrogen or methyl;

HET-2 is selected from azetidinyl, morpholino, morpholinyl, piperidinyl, piperazinyl, 3-oxopiperazinyl, thiomorpholinyl, pyrrolidinyl, pyrrolidonyl, 2,5-dioxopyrrolidinyl, 1,1-dioxotetrahydrothienyl, 2-oxazolidinonyl, 2-oxotetrahydrofuranyl, tetrahydrofuranyl, tetrahydropyranyl, 1,1-dioxothiomorpholino, 1,3-dioxolanyl, 2-oxoimidazolidinyl, 2,4-dioxoimidazolidinyl, pyranyl and 4-pyridonyl; wherein HET-2 is optionally substituted by R⁷; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from thiazolyl, isothiazolyl, thiadiazolyl, pyrazolyl, imidazolyl, oxazolyl, isoxazolyl and oxadiazolyl;

R² is HET-2;

R³ is halo or trifluoromethyl;

R⁵ is hydrogen or methyl;

HET-2 is selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, pyrrolyl, 1,2,4-triazolyl and 1,2,3-triazolyl; wherein HET-2 is optionally substituted by R⁷; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from pyridyl, pyrazinyl, pyridazinyl and pyrimidinyl;

R² is HET-2;

R³ is halo or trifluoromethyl;

R⁵ is hydrogen or methyl;

HET-2 is selected from azetidinyl, morpholino, morpholinyl, piperidinyl, piperazinyl, 3-oxopiperazinyl, thiomorpholinyl, pyrrolidinyl, pyrrolidonyl, 2,5-dioxopyrrolidinyl, 1,1-dioxotetrahydrothienyl, 2-oxazolidinonyl, 2-oxotetrahydrofuranyl, tetrahydrofuranyl, tetrahydropyranyl, 1,1-dioxothiomorpholino, 1,3-dioxolanyl, 2-oxoimidazolidinyl, 2,4-dioxoimidazolidinyl, pyranyl and 4-pyridonyl; wherein HET-2 is optionally substituted by R⁷; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from pyridyl, pyrazinyl, pyridazinyl and pyrimidinyl;

R² is HET-2;

R³ is halo or trifluoromethyl;

R⁵ is hydrogen or methyl;

HET-2 is selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, pyrrolyl, 1,2,4-triazolyl and 1,2,3-triazolyl; wherein HET-2 is optionally substituted by R⁷; and

R⁷ is selected from —OR⁵ and (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from thiazolyl, isothiazolyl, thiadiazolyl, pyrazolyl, imidazolyl, oxazolyl, isoxazolyl and oxadiazolyl and is optionally substituted by a group R⁶;

R² is HET-2;

R³ is halo or trifluoromethyl;

R⁶ is selected from methyl, ethyl, bromo, chloro, fluoro, hydroxymethyl, methoxymethyl, aminomethyl, N-methylaminomethyl, and dimethylaminomethyl;

HET-2 is selected from azetidinyl, morpholino, morpholinyl, piperidinyl, piperazinyl, 3-oxopiperazinyl, thiomorpholinyl, pyrrolidinyl, pyrrolidonyl, 2,5-dioxopyrrolidinyl, 1,1-dioxotetrahydrothienyl, 2-oxazolidinonyl, 2-oxotetrahydrofuranyl, tetrahydrofuranyl, tetrahydropyranyl, 1,1-dioxothiomorpholino, 1,3-dioxolanyl, 2-oxoimidazolidinyl, 2,4-dioxoimidazolidinyl, pyranyl and 4-pyridonyl; wherein HET-2 is optionally substituted by R⁷; and

R⁷ is (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from thiazolyl, isothiazolyl, thiadiazolyl, pyrazolyl, imidazolyl, oxazolyl, isoxazolyl and oxadiazolyl and is optionally substituted by a group R⁶;

R² is HET-2;

R³ is halo or trifluoromethyl;

R⁶ is selected from methyl, ethyl, bromo, chloro, fluoro, hydroxymethyl, methoxymethyl, aminomethyl, N-methylaminomethyl, and dimethylaminomethyl;

HET-2 is selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, pyrrolyl, 1,2,4-triazolyl and 1,2,3-triazolyl; wherein HET-2 is optionally substituted by R⁷; and

R⁷ is (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from pyridyl, pyrazinyl, pyridazinyl and pyrimidinyl and is optionally substituted by a group R⁶;

R² is HET-2;

R³ is halo or trifluoromethyl;

R⁶ is selected from methyl, ethyl, bromo, chloro, fluoro, hydroxymethyl, methoxymethyl, aminomethyl, N-methylaminomethyl, and dimethylaminomethyl;

HET-2 is selected from azetidinyl, morpholino, morpholinyl, piperidinyl, piperazinyl, 3-oxopiperazinyl, thiomorpholinyl, pyrrolidinyl, pyrrolidonyl, 2,5-dioxopyrrolidinyl, 1,1-dioxotetrahydrothienyl, 2-oxazolidinonyl, 2-oxotetrahydrofuranyl, tetrahydrofuranyl, tetrahydropyranyl, 1,1-dioxothiomorpholino, 1,3-dioxolanyl, 2-oxoimidazolidinyl, 2,4-dioxoimidazolidinyl, pyranyl and 4-pyridonyl; wherein HET-2 is optionally substituted by R⁷; and

R⁷ is (1-4C)alkyl;

or a salt, pro-drug or solvate thereof.

In a further aspect of the invention is provided a compound of the formula (I) as hereinbefore defined wherein

R¹ is hydroxymethyl;

m is 1 and n is 0 or 1;

HET-1 is selected from pyridyl, pyrazinyl, pyridazinyl and pyrimidinyl and is optionally substituted by a group R⁶;

R² is HET-2;

R³ is halo or trifluoromethyl;

R⁶ is selected from methyl, ethyl, bromo, chloro, fluoro, hydroxymethyl, methoxymethyl, aminomethyl, N-methylaminomethyl, and dimethylaminomethyl;

HET-2 is selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, pyrrolyl, 1,2,4-triazolyl and 1,2,3-triazolyl; wherein HET-2 is optionally substituted by R⁷; and

R⁷ is (1-4C)alkyl;

or a salt, pro-drug or solvate thereof.

In a further aspect of the invention there is provided a compound of the formula (I) as hereinbefore defined wherein

R¹ is hydroxymethyl;

m is 1 and n is 0 or 1;

HET-1 is 3-pyrazolyl, substituted on a nitrogen atom by methyl or ethyl;

R² is selected from dimethylaminocarbonyl, N-azetidinylcarbonyl, N-pyrrolidinylcarbonyl, methylsulfonyl and ethylsulfonyl;

R³ is fluoro or chloro;

or a salt, pro-drug or solvate thereof.

Further preferred compounds of the invention are each of the Examples, each of which provides a further independent aspect of the invention. In further aspects, the present invention also comprises any two or more compounds of the Examples.

In one aspect, particular compounds of the invention comprise any one or more of:
3-[4-(azetidin-1-ylcarbonyl)-2-fluorophenoxy]-5-{[(1S)-1-(hydroxymethyl)propyl]oxy}-N-(1-methyl-1H-pyrazol-3-yl)benzamide;
3-[2-chloro-4-(pyrrolidin-1-ylcarbonyl)phenoxy]-5-{[(1S)-1-(hydroxymethyl)propyl]oxy}-N-(1-methyl-1H-pyrazol-3-yl)benzamide;
3-fluoro-4-(3-{[(1S)-1-(hydroxymethyl)propyl]oxy}-5-{[(1-methyl-1H-pyrazol-3-yl)amino]carbonyl}phenoxy)-N,N-dimethylbenzamide;
3-[4-(azetidin-1-ylcarbonyl)-2-chlorophenoxy]-5-{[(1S)-1-(hydroxymethyl)propyl]oxy}-N-(1-methyl-1H-pyrazol-3-yl)benzamide;
3-[2-fluoro-4-(methylsulfonyl)phenoxy]-5-{[(1S)-1-(hydroxymethyl)propyl]oxy}-N-(1-methyl-1H-pyrazol-3-yl)benzamide;
3-[2-chloro-4-(methylsulfonyl)phenoxy]-5-{[(1S)-1-(hydroxymethyl)propyl]oxy}-N-(1-methyl-1H-pyrazol-3-yl)benzamide;
3-[4-(ethylsulfonyl)-2-fluorophenoxy]-5-{[(1S)-1-(hydroxymethyl)propyl]oxy}-N-(1-methyl-1H-pyrazol-3-yl)benzamide;
3-chloro-4-(3-{[(1S)-1-(hydroxymethyl)propyl]oxy}-5-{[(1-methyl-1H-pyrazol-3-yl)amino]carbonyl}phenoxy)-N,N-dimethylbenzamide;
3-[2-fluoro-4-(pyrrolidin-1-ylcarbonyl)phenoxy]-5-{[(1S)-1-(hydroxymethyl)propyl]oxy}-N-(1-methyl-1H-pyrazol-3-yl)benzamide;
N-(1-ethyl-1H-pyrazol-3-yl)-3-[2-fluoro-4-(methylsulfonyl)phenoxy]-5-{[(1S)-1-(hydroxymethyl)propyl]oxy}benzamide;
3-chloro-4-(3-{[(1-ethyl-1H-pyrazol-3-yl)amino]carbonyl}-5-{[(1S)-1-(hydroxymethyl)propyl]oxy}phenoxy)-N,N-dimethylbenzamide;
3-{[(1S)-1-(hydroxymethyl)propyl]oxy}-N-(1-methyl-1H-pyrazol-3-yl)-5-[4-(pyrrolidin-1-ylcarbonyl)phenoxy]benzamide;
3-[4-(azetidin-1-ylcarbonyl)phenoxy]-5-{[(1S)-1-(hydroxymethyl)propyl]oxy}-N-(1-methyl-1H-pyrazol-3-yl)benzamide;
3-{4-[(dimethylamino)carbonyl]phenoxy}-N-(1-ethyl-1H-pyrazol-3-yl)-5-{[(1S)-1-(hydroxymethyl)propyl]oxy}benzamide; and
3-{4-[(dimethylamino)carbonyl]phenoxy}-5-{[(1S)-1-(hydroxymethyl)propyl]oxy}-N-(1-methyl-1H-pyrazol-3-yl)benzamide;
   or a salt, pro-drug or solvate thereof.

In a further aspect, particular compounds of the invention comprise any one or more of:
3-[4-(azetidin-1-ylcarbonyl)-2-fluorophenoxy]-5-{[(1S)-1-(hydroxymethyl)propyl]oxy}-N-(1-methyl-1H-pyrazol-3-yl)benzamide;
3-[2-chloro-4-(pyrrolidin-1-ylcarbonyl)phenoxy]-5-{[(1S)-1-(hydroxymethyl)propyl]oxy}-N-(1-methyl-1H-pyrazol-3-yl)benzamide;
3-[4-(azetidin-1-ylcarbonyl)-2-chlorophenoxy]-5-{[(1S)-1-(hydroxymethyl)propyl]oxy}-N-(1-methyl-1H-pyrazol-3-yl)benzamide;
3-[2-fluoro-4-(pyrrolidin-1-ylcarbonyl)phenoxy]-5-{[(1S)-1-(hydroxymethyl)propyl]oxy}-N-(1-methyl-1H-pyrazol-3-yl)benzamide;
3-{[(1S)-1-(hydroxymethyl)propyl]oxy}-N-(1-methyl-1H-pyrazol-3-yl)-5-[4-(pyrrolidin-1-ylcarbonyl)phenoxy]benzamide; and
3-[4-(azetidin-1-ylcarbonyl)phenoxy]-5-{[(1S)-1-(hydroxymethyl)propyl]oxy}-N-(1-methyl-1H-pyrazol-3-yl)benzamide;
   or a salt, pro-drug or solvate thereof.

The compounds of the invention may be administered in the form of a pro-drug. A pro-drug is a bioprecursor or pharmaceutically acceptable compound being degradable in the body to produce a compound of the invention (such as an ester or amide of a compound of the invention, particularly an in-vivo hydrolysable ester). Various forms of prodrugs are known in the art. For examples of such prodrug derivatives, see:

a) Design of Prodrugs, edited by H. Bundgaard, (Elsevier, 1985) and Methods in Enzymology, Vol. 42, p. 309-396, edited by K. Widder, et al. (Academic Press, 1985);

b) A Textbook of Drug Design and Development, edited by Krogsgaard-Larsen;

c) H. Bundgaard, Chapter 5 “Design and Application of Prodrugs”, by H. Bundgaardp. 113-191 (1991);

d) H. Bundgaard, Advanced Drug Delivery Reviews, 8, 1-38 (1992);

e) H. Bundgaard, et al., Journal of Pharmaceutical Sciences, 77, 285 (1988); and

f) N. Kakeya, et al., Chem Pharm Bull, 32, 692 (1984).

The contents of the above cited documents are incorporated herein by reference.

Examples of pro-drugs are as follows. An in-vivo hydrolysable ester of a compound of the invention containing a carboxy or a hydroxy group is, for example, a pharmaceutically-acceptable ester which is hydrolysed in the human or animal body to produce the parent acid or alcohol. Suitable pharmaceutically-acceptable esters for carboxy include C₁ to C₆alkoxymethyl esters for example methoxymethyl, C₁ to C₆alkanoyloxymethyl esters for example pivaloyloxymethyl, phthalidyl esters, C₃ to C₈cycloalkoxycarbonyloxyC₁ to C₆alkyl esters for example 1-cyclohexylcarbonyloxyethyl; 1,3-dioxolen-2-onylmethyl esters, for example 5-methyl-1,3-dioxolen-2-onylmethyl; and C₁₋₆alkoxycarbonyloxyethyl esters.

An in-vivo hydrolysable ester of a compound of the invention containing a hydroxy group includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxy-methoxy. A selection of in-vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

A suitable pharmaceutically-acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. It will be understood that an acid addition salt may be formed with any sufficiently basic group which may for example be in HET-1 or may for example be a substituent R². In addition a suitable pharmaceutically-acceptable salt of a benzoxazinone derivative of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

A further feature of the invention is a pharmaceutical composition comprising a compound of Formula (I) as defined above, or a salt, solvate or prodrug thereof, together with a pharmaceutically-acceptable diluent or carrier.

According to another aspect of the invention there is provided a compound of Formula (I) as defined above for use as a medicament.

Further according to the invention there is provided a compound of Formula (I) for use in the preparation of a medicament for treatment of a disease mediated through GLK, in particular type 2 diabetes.

The compound is suitably formulated as a pharmaceutical composition for use in this way.

According to another aspect of the present invention there is provided a method of treating GLK mediated diseases, especially diabetes, by administering an effective amount of a compound of Formula (I) or salt, solvate or pro-drug thereof, to a mammal in need of such treatment.

Specific diseases which may be treated by a compound or composition of the invention include: blood glucose lowering in Type 2 Diabetes Mellitus without a serious risk of hypoglycaemia (and potential to treat type 1), dyslipidemia, obesity, insulin resistance, metabolic syndrome X, impaired glucose tolerance.

As discussed above, thus the GLK/GLKRP system can be described as a potential “Diabesity” target (of benefit in both Diabetes and Obesity). Thus, according to another aspect of the invention there if provided the use of a compound of Formula (I) or salt, solvate or pro-drug thereof, in the preparation of a medicament for use in the combined treatment or prevention of diabetes and obesity.

According to another aspect of the invention there if provided the use of a compound of Formula (I) or salt, solvate or pro-drug thereof, in the preparation of a medicament for use in the treatment or prevention of obesity.

According to a further aspect of the invention there is provided a method for the combined treatment of obesity and diabetes by administering an effective amount of a compound of Formula (I) or salt, solvate or pro-drug thereof, to a mammal in need of such treatment.

According to a further aspect of the invention there is provided a method for the treatment of obesity by administering an effective amount of a compound of Formula (I) or salt, solvate or pro-drug thereof, to a mammal in need of such treatment.

Compounds of the invention may be particularly suitable for use as pharmaceuticals, for example because of favourable physical and/or pharmacokinetic properties and/or toxicity profile and/or potency.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing). Generally, dosage forms suitable for oral use are preferred.

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound of the Formula (I) will naturally vary according to the nature and severity of the conditions, the age and sex of the animal or patient and the route of administration, according to well known principles of medicine.

In using a compound of the Formula (I) for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred.

The elevation of GLK activity described herein may be applied as a sole therapy or in combination with one or more other substances and/or treatments for the indication being treated. Such conjoint treatment may be achieved by way of the simultaneous, sequential or separate administration of the individual components of the treatment. Simultaneous treatment may be in a single tablet or in separate tablets. For example in the treatment of diabetes mellitus, chemotherapy may include the following main categories of treatment:

1) Insulin and insulin analogues;

2) Insulin secretagogues including sulphonylureas (for example glibenclamide, glipizide), prandial glucose regulators (for example repaglinide, nateglinide);

3) Agents that improve incretin action (for example dipeptidyl peptidase IV inhibitors, and GLP-1 agonists);

4) Insulin sensitising agents including PPARgamma agonists (for example pioglitazone and rosiglitazone), and agents with combined PPARalpha and gamma activity;

5) Agents that modulate hepatic glucose balance (for example metformin, fructose 1, 6 bisphosphatase inhibitors, glycogen phopsphorylase inhibitors, glycogen synthase kinase inhibitors);

6) Agents designed to reduce the absorption of glucose from the intestine (for example acarbose);

7) Agents that prevent the reabsorption of glucose by the kidney (SGLT inhibitors);

8) Agents designed to treat the complications of prolonged hyperglycaemia (for example aldose reductase inhibitors);

9) Anti-obesity agents (for example sibutramine and orlistat);

10) Anti-dyslipidaemia agents such as, HMG-CoA reductase inhibitors (eg statins); PPARα agonists (fibrates, eg gemfibrozil); bile acid sequestrants (cholestyramine); cholesterol absorption inhibitors (plant stanols, synthetic inhibitors); bile acid absorption inhibitors (IBATi) and nicotinic acid and analogues (niacin and slow release formulations);

11) Antihypertensive agents such as, β blockers (eg atenolol, inderal); ACE inhibitors (eg lisinopril); Calcium antagonists (eg. nifedipine); Angiotensin receptor antagonists (eg candesartan), α antagonists and diuretic agents (eg. furosemide, benzthiazide);

12) Haemostasis modulators such as, antithrombotics, activators of fibrinolysis and antiplatelet agents; thrombin antagonists; factor Xa inhibitors; factor VIIa inhibitors); antiplatelet agents (eg. aspirin, clopidogrel); anticoagulants (heparin and Low molecular weight analogues, hirudin) and warfarin;

13) Agents which antagonise the actions of glucagon; and

14) Anti-inflammatory agents, such as non-steroidal anti-inflammatory drugs (eg. aspirin) and steroidal anti-inflammatory agents (eg. cortisone).

According to another aspect of the present invention there is provided individual compounds produced as end products in the Examples set out below and salts, solvates and pro-drugs thereof.

A compound of the invention, or a salt thereof, may be prepared by any process known to be applicable to the preparation of such compounds or structurally related compounds. Functional groups may be protected and deprotected using conventional methods. For examples of protecting groups such as amino and carboxylic acid protecting groups (as well as means of formation and eventual deprotection), see T. W. Greene and P. G. M. Wuts, “Protective Groups in Organic Synthesis”, Second Edition, John Wiley & Sons, New York, 1991.

Processes for the synthesis of compounds of Formula (I) are provided as a further feature of the invention. Thus, according to a further aspect of the invention there is provided a process for the preparation of a compound of Formula (I), which comprises a process a) to e) (wherein the variables are as defined hereinbefore for compounds of Formula (I) unless otherwise defined):
(a) reaction of an acid of Formula (III) or activated derivative thereof with a compound of Formula (IV), wherein R¹ is hydroxymethyl or a protected version thereof; 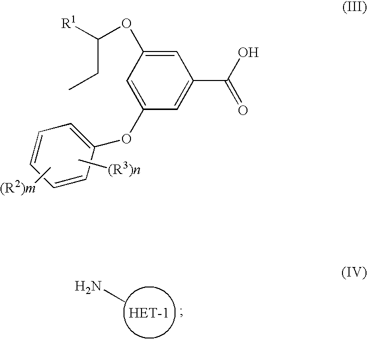
   or
(b) reaction of a compound of Formula (V) with a compound of Formula (VI), 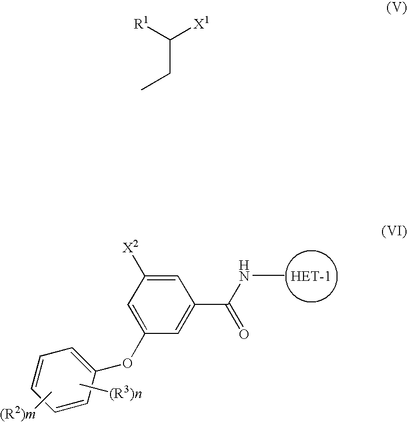
   wherein X¹ is a leaving group and X² is a hydroxyl group or X¹ is a hydroxyl group and X² is a leaving group, and wherein R¹ is hydroxymethyl or a protected version thereof; process (b) could also be accomplished using the intermediate ester Formula (VII), wherein P¹ is a protecting group as hereinafter described, followed by ester hydrolysis and amide formation by procedures described elsewhere and well known to those skilled in the art; 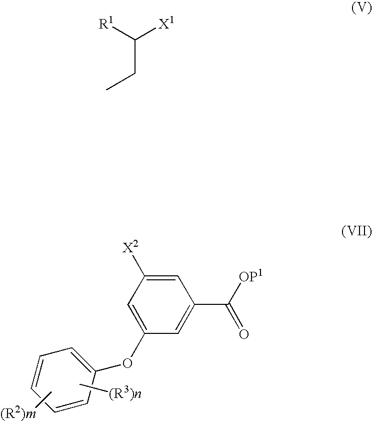
   or
(c) reaction of a compound of Formula (VIII) with a compound of Formula (IX) 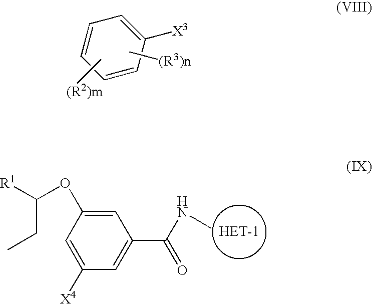
   wherein X³ is a leaving group or an organometallic reagent and X⁴ is a hydroxyl group or X³ is a hydroxyl group and X⁴ is a leaving group or an organometallic reagent, and wherein R¹ is hydroxymethyl or a protected version thereof;
   process (c) could also be accomplished using the intermediate ester Formula (X), followed by ester hydrolysis and amide formation by procedures described elsewhere and well known to those skilled in the art; 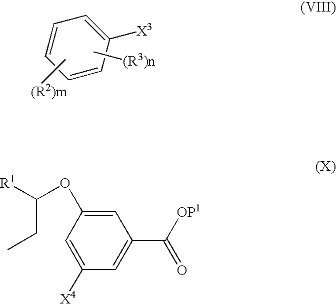
   or
(d) reaction of a compound of Formula (XI) with a compound of Formula (XII), 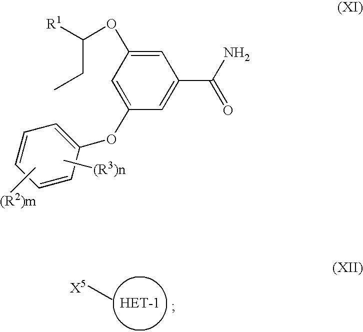
   wherein X⁵ is a leaving group; and wherein R¹ is hydroxymethyl or a protected version thereof; or
e) reaction of a compound of formula (XIII) 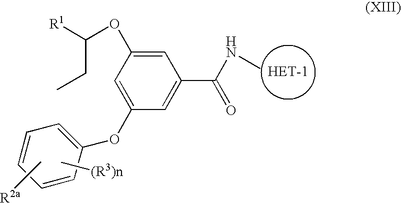
   wherein R^{2a} is a precursor to R², such as a carboxylic acid, ester or anhydride (for R² ]-CONR⁴R⁵) or the sulfonic acid equivalents (for R² is —SO²NR⁴R⁵); with an amine of formula —NR⁴R⁵;
   and thereafter, if necessary:
   i) converting a compound of Formula (I) into another compound of Formula (I);
   ii) removing any protecting groups; and/or
   iii) forming a salt, pro-drug or solvate thereof.

Suitable leaving groups X¹ to X⁵ for processes b) to d) are any leaving group known in the art for these types of reactions, for example halo, alkoxy, trifluoromethanesulfonyloxy, methanesulfonyloxy, or p-toluenesulfonyloxy; or a group (such as a hydroxy group) that may be converted into a leaving group (such as an oxytriphenylphosphonium group) in situ.

Suitable values for R¹ as a protected hydroxy group are any suitable protected hydroxy group known in the art, for example simple ethers such as a methyl ether, or silylethers such as —OSi[(1-4C)alkyl]₃ (wherein each (1-4C)alkyl group is independently selected from methyl, ethyl, propyl, isopropyl, and tertbutyl). Examples of such trialkylsilyl groups are trimethylsilyl, triethylsilyl, triisopropylsilyl and tert-butyldimethylsilyl. Further suitable silyl ethers are those containing phenyl and substituted phenyl groups, such as —Si(PhMe₂) and

—Si(TolMe₂) (wherein Tol=methylbenzene). Further suitable values for hydroxy protecting groups are given hereinafter.

Compounds of Formulae (III) to (XII) are commercially available, or are known in the art, or may be made by processes known in the art, for example as shown in the accompanying Examples. For further information on processes for making such compounds, we refer to our PCT publications WO 03/000267, WO 03/015774 and WO 03/000262 and references therein. In general it will be appreciated that any aryl-O or alkyl-O bond may be formed by nucleophilic substitution or metal catalysed processes, optionally in the presence of a suitable base.

Compounds of Formula (XIII) may be made by processes such as those shown in processes a) to d) and/or by those processes mentioned above for compounds of formulae (III) to (XII).

Examples of conversions of a compound of Formula (I) into another compound of Formula (I), well known to those skilled in the art, include functional group interconversions such as hydrolysis, hydrogenation, hydrogenolysis, oxidation or reduction, and/or further functionalisation by standard reactions such as amide or metal-catalysed coupling, or nucleophilic displacement reactions. An example would be removal of an R³=chloro substituent, for example by reaction with hydrogen at atmospheric or elevated pressure, in a suitable solvent such as THF/methanol or ethanol.

It will be understood that substituents R⁸, R⁶ and/or R⁷ may be introduced into the molecule at any convenient point in the synthetic sequence or may be present in the starting materials. A precursor to one of these substituents may be present in the molecule during the process steps a) to e) above, and then be transformed into the desired substituent as a final step to form the compound of formula (I); followed where necessary by

i) converting a compound of Formula (I) into another compound of Formula (I);

ii) removing any protecting groups; and/or

iii) forming a salt or pro-drug thereof.

Specific reaction conditions for the above reactions are as follows, wherein when P¹ is a protecting group P¹ is preferably C₁₋₄alkyl, for example methyl or ethyl:

Process a)—coupling reactions of amino groups with carboxylic acids to form an amide are well known in the art. For example,

(i) using an appropriate coupling reaction, such as a carbodiimide coupling reaction performed with EDAC (1-(3-dimethylaminopropyl)-3-ethylcarbodiimide hydrochloride) in the presence of dimethylaminopyridine in a suitable solvent such as dichloromethane (DCM), chloroform or dimethylformamide (DMF) at room temperature; or

(ii) reaction in which the carboxylic group is activated to an acid chloride by reaction with oxalyl chloride in the presence of a suitable solvent such as DCM. The acid chloride can then be reacted with a compound of Formula (IV) in the presence of a base, such as triethylamine or pyridine, in a suitable solvent such as DCM or pyridine at a temperature between 0° C. and 80° C.

Process b)—compounds of Formula (V) and (VI) can be reacted together in a suitable solvent, such as DMF or tetrahydrofuran (THF), with a base such as sodium hydride or potassium tert-butoxide, at a temperature in the range 0 to 200° C., optionally using microwave heating or metal catalysis such as palladium(II)acetate, palladium on carbon, copper(II)acetate or copper(I)iodide; alternatively, compounds of Formula (V) and (VI) can be reacted together in a suitable solvent, such as THF or DCM, with a suitable phosphine such as triphenylphosphine, and azodicarboxylate such as diethylazodicarboxylate; process b) could also be carried out using a precursor to the ester of formula (VII) such as an aryl-nitrile or trifluoromethyl derivative, followed by conversion to a carboxylic acid and amide formation as previously described;

Process c)—compounds of Formula (VIII) and (IX) can be reacted together in a suitable solvent, such as DMF or THF, with a base such as sodium hydride or potassium tert-butoxide, at a temperature in the range 0 to 200° C., optionally using microwave heating or metal catalysis such as palladium(II)acetate, palladium on carbon, copper(II)acetate or copper(I)iodide; process b) could also be carried out using a precursor to the ester of formula (X) such as an aryl-nitrile or trifluoromethyl derivative, followed by conversion to a carboxylic acid and amide formation as previously described;

compounds of the formula (VIII) are commercially available or can be prepared from commercially available materials by processes well known to those skilled in the art, for example functional group interconversions (such as hydrolysis, hydrogenation, hydrogenolysis, oxidation or reduction), and/or further functionalisation and/or cyclisation by standard reactions (such as amide or sulphonamide or metal-catalysed coupling, or nucleophilic displacement or electrophilic substitution reactions);

Process d)—reaction of a compound of Formula (XI) with a compound of Formula (XII) can be performed in a polar solvent, such as DMF or a non-polar solvent such as THF with a strong base, such as sodium hydride or potassium tert-butoxide at a temperature between 0 and 200° C., optionally using microwave heating or metal catalysis, such as palladium(II)acetate, palladium on carbon, copper(II)acetate or copper(I)iodide;

Process e)—coupling reactions of amino groups with carboxylic or sulfonic acids or acid derivatives to form an amide are well known in the art and are described above for Process a).

Certain intermediates of formula (III), (VI), (VII), (IX) and/or (XI) are believed to be novel and comprise an independent aspect of the invention.

Certain intermediates of formula (III), (IX) and/or (XI) wherein R¹ is hydroxymethyl, or a trialkylsilylether are believed to be novel and comprise an independent aspect of the invention.

Certain intermediates of formula (XIII) are believed to be novel and comprise an independent aspect of the invention.

During the preparation process, it may be advantageous to use a protecting group for a functional group within the molecule. Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Specific examples of protecting groups are given below for the sake of convenience, in which “lower” signifies that the group to which it is applied preferably has 1-4 carbon atoms. It will be understood that these examples are not exhaustive. Where specific examples of methods for the removal of protecting groups are given below these are similarly not exhaustive. The use of protecting groups and methods of deprotection not specifically mentioned is of course within the scope of the invention.

A carboxy protecting group may be the residue of an ester-forming aliphatic or araliphatic alcohol or of an ester-forming silanol (the said alcohol or silanol preferably containing 1-20 carbon atoms). Examples of carboxy protecting groups include straight or branched chain (1-12C)alkyl groups (e.g. isopropyl, t-butyl); lower alkoxy lower alkyl groups (e.g. methoxymethyl, ethoxymethyl, isobutoxymethyl; lower aliphatic acyloxy lower alkyl groups, (e.g. acetoxymethyl, propionyloxymethyl, butyryloxymethyl, pivaloyloxymethyl); lower alkoxycarbonyloxy lower alkyl groups (e.g. 1-methoxycarbonyloxyethyl, 1-ethoxycarbonyloxyethyl); aryl lower alkyl groups (e.g. p-methoxybenzyl, o-nitrobenzyl, p-nitrobenzyl, benzhydryl and phthalidyl); tri(lower alkyl)silyl groups (e.g. trimethylsilyl and t-butyldimethylsilyl); tri(lower alkyl)silyl lower alkyl groups (e.g. trimethylsilylethyl); and (2-6C)alkenyl groups (e.g. allyl and vinylethyl).

Methods particularly appropriate for the removal of carboxyl protecting groups include for example acid-, base-, metal- or enzymically-catalysed hydrolysis. Hydrogenation may also be used.

Examples of hydroxy protecting groups include methyl, lower alkenyl groups (e.g. allyl); lower alkanoyl groups (e.g. acetyl); lower alkoxycarbonyl groups (e.g. t-butoxycarbonyl); lower alkenyloxycarbonyl groups (e.g. allyloxycarbonyl); aryl lower alkoxycarbonyl groups (e.g. benzoyloxycarbonyl, p-methoxybenzyloxycarbonyl, o-nitrobenzyloxycarbonyl, p-nitrobenzyloxycarbonyl); tri lower alkyl/arylsilyl groups (e.g. trimethylsilyl, t-butyldimethylsilyl, t-butyldiphenylsilyl); aryl lower alkyl groups (e.g. benzyl) groups; and triaryl lower alkyl groups (e.g. triphenylmethyl).

Examples of amino protecting groups include formyl, aralkyl groups (e.g. benzyl and substituted benzyl, e.g. p-methoxybenzyl, nitrobenzyl and 2,4-dimethoxybenzyl, and triphenylmethyl); di-p-anisylmethyl and furylmethyl groups; lower alkoxycarbonyl (e.g. t-butoxycarbonyl); lower alkenyloxycarbonyl (e.g. allyloxycarbonyl); aryl lower alkoxycarbonyl groups (e.g. benzyloxycarbonyl, p-methoxybenzyloxycarbonyl, o-nitrobenzyloxycarbonyl, p-nitrobenzyloxycarbonyl; trialkylsilyl (e.g. trimethylsilyl and t-butyldimethylsilyl); alkylidene (e.g. methylidene); benzylidene and substituted benzylidene groups.

Methods appropriate for removal of hydroxy and amino protecting groups include, for example, acid-, base, metal- or enzymically-catalysed hydrolysis, or photolytically for groups such as o-nitrobenzyloxycarbonyl, or with fluoride ions for silyl groups, or catalytic hydrogenation. For example, methylether protecting groups for hydroxy groups may be removed by trimethylsilyliodide. A tert-butyl ether protecting group for a hydroxy group may be removed by hydrolysis, for example by use of hydrochloric acid in methanol.

Examples of protecting groups for amide groups include aralkoxymethyl (e.g. benzyloxymethyl and substituted benzyloxymethyl); alkoxymethyl (e.g. methoxymethyl and trimethylsilylethoxymethyl); tri alkyl/arylsilyl (e.g. trimethylsilyl, t-butyldimethylsily, t-butyldiphenylsilyl); tri alkyl/arylsilyloxymethyl (e.g. 1-butyldimethylsilyloxymethyl, t-butyldiphenylsilyloxymethyl); 4-alkoxyphenyl (e.g. 4-methoxyphenyl); 2,4-di(alkoxy)phenyl (e.g. 2,4-dimethoxyphenyl); 4-alkoxybenzyl (e.g. 4-methoxybenzyl); 2,4-di(alkoxy)benzyl (e.g. 2,4-di(methoxy)benzyl); and alk-1-enyl (e.g. allyl, but-1-enyl and substituted vinyl e.g. 2-phenylvinyl).

Aralkoxymethyl, groups may be introduced onto the amide group by reacting the latter group with the appropriate aralkoxymethyl chloride, and removed by catalytic hydrogenation. Alkoxymethyl, tri alkyl/arylsilyl and tri alkyl/silyloxymethyl groups may be introduced by reacting the amide with the appropriate chloride and removing with acid; or in the case of the silyl containing groups, fluoride ions. The alkoxyphenyl and alkoxybenzyl groups are conveniently introduced by arylation or alkylation with an appropriate halide and removed by oxidation with ceric ammonium nitrate. Finally alk-1-enyl groups may be introduced by reacting the amide with the appropriate aldehyde and removed with acid.

The following examples are for illustration purposes and are not intended to limit the scope of this application. Each exemplified compound represents a particular and independent aspect of the invention. In the following non-limiting examples, unless otherwise stated:
(i) evaporations were carried out by rotary evaporation in vacuo and work-up procedures were carried out after removal of residual solids such as drying agents by filtration;
(ii) operations were carried out at room temperature, that is in the range 18-25° C. and under an atmosphere of an inert gas such as argon or nitrogen unless otherwise stated;
(iii) yields are given for illustration only and are not necessarily the maximum attainable;
(iv) the structures of the end-products of the Formula (I) were confirmed by nuclear (generally proton) magnetic resonance (NMR) with a field strength (for proton) of 300 MHz (generally using a Varian Gemini 2000) or 400 MHz (generally using a Bruker Avance DPX400), unless otherwise stated, and mass spectral techniques; proton magnetic resonance chemical shift values were measured on the delta scale and peak multiplicities are shown as follows: s, singlet; d, doublet; t, triplet; m, multiplet; br, broad; q, quartet, quin, quintet;
(v) intermediates were not generally fully characterised and purity was assessed by thin layer chromatography (TLC), high-performance liquid chromatography (HPLC), infra-red (IR) or NMR analysis
(vi) Purification by chromatography generally refers to flash column chromatography, on silica unless otherwise stated. Column chromatography was generally carried out using prepacked silica cartridges (from 4 g up to 400 g) such as Redisep™ (available, for example, from Presearch Ltd, Hitchin, Herts, UK) or Biotage (Biotage UK Ltd, Hertford, Herts, UK), eluted using a pump and fraction collector system. Purification by Solid Phase Extraction (SPE) methods generally refers to the use of chromatography cartridges packed with SPE materials such as ISOLUTE® SCX-2 columns (available, for example, From International Sorbent Technology Ltd, Dryffryn Business Park, Hengoed, Mid Glamorgan, UK);
(vii) Mass spectra (MS) data was generated on an LCMS system where the HPLC component comprised generally either a Agilent 1100 or Waters Alliance HT (2790 & 2795) equipment and was run on a Phemonenex Gemini C18 5 μm, 50×2 mm column (or similar) eluting with either acidic eluent (for example, using a gradient between 0-95% water/acetonitrile with 5% of a 1% formic acid in 50:50 water:acetonitrile (v/v) mixture;
   or using an equivalent solvent system with methanol instead of acetonitrile), or basic eluent (for example, using a gradient between 0-95% water/acetonitrile with 5% of a 0.1% 880 Ammonia in acetonitrile mixture); and the MS component comprised generally a Waters ZQ spectrometer. Chromatograms for Electrospray (ESI) positive and negative Base Peak Intensity, and UV Total Absorption Chromatogram from 220-300 nm, are generated and values for m/z are given; generally, only ions which indicate the parent mass are reported and unless otherwise stated the value quoted is (M−H)⁻;

(viii) Suitable microwave reactors include “Smith Creator”, “CEM Explorer”, “Biotage Initiator sixty” and “Biotage Initiator eight”.

Abbreviations

| | |
|---|---|
| CDCl₃ | deuterochloroform; |
| DCM | dichloromethane; |
| DEAD | diethylazodicarboxylate; |
| DIAD | diisopropylazodicarboxylate; |
| DIPEA | N,N-Diisopropylethylamine; |
| DMSO | dimethyl sulfoxide; |
| DMF | dimethylformamide; |
| HATU | O-(7-azabenzotriazol-1-yl)-N,N,N′,N′- |
| | tetramethyluronium hexafluorophosphate; |
| HPLC | high pressure liquid chromatography |
| HPMC | hydroxypropylmethylcellulose; |
| LCMS | liquid chromatography/mass spectroscopy; |
| NMR | nuclear magnetic resonance spectroscopy; |
| pH | −log₁₀[hydrogen ion] |
| RT | room temperature; |
| THF | tetrahydrofuran; |
| TFA | trifluoroacetic acid |

All compound names were derived using ACD NAME computer package.

### EXAMPLE 1

### 3-[4-(Azetidin-1-ylcarbonyl)-2-fluorophenoxy]-5-{[(1S)-1-(hydroxymethyl)propyl]oxy}-N-(1-methyl-1H-pyrazol-3-yl)benzamide

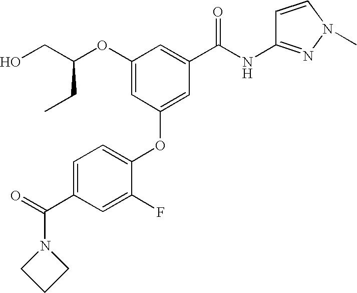 Potassium carbonate (226 mg, 1.63 mmol) was added to a solution of 3-hydroxy-5-{[(1S)-1-(hydroxymethyl)propyl]oxy}-N-(1-methyl-1H-pyrazol-3-yl)benzamide (Intermediate 2, 250 mg, 0.82 mmol) and 1-(3,4-difluorobenzoyl)azetidine (Intermediate 1, 169 mg, 0.86 mmol) in acetonitrile (5 mL). The resulting mixture was subjected to microwave heating at 160° C. for 5 hours. The mixture was filtered then concentrated in vacuo. The residue taken up in ethyl acetate (30 mL), washed twice with water (5 mL) and once with brine (5 mL), dried (MgSO₄), filtered and evaporated and purified by column chromatography (eluting with 85% ethyl acetate in isohexane) to give the title compound as a colourless foam (157 mg, 40%). ¹H NMR δ (CDCl₃): 0.95 (t, 3H), 1.6-1.8 (m, 2H), 2.15 (b, 1H), 2.4 (m, 2H), 3.8 (m, 5H), 4.2-4.4 (m, 5H), 6.8 (s, 2H), 7.0 (d, 1H), 7.1 (s, 1H), 7.3 (m, 2H), 7.5 (d, 1H), 7.8 (s, 1H), 9.0 (s, 1H): m/z 483 (M+H)⁺

### Intermediate 1: 1-(3,4-Difluorobenzoyl)azetidine

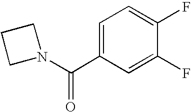 Oxalyl chloride (1.05 mL, 12.0 mmol) was added to a solution of 3,4-difluorobenzoic acid (1.58 g, 10 mmol) in DCM (50 mL) containing DMF (1 drop). The reaction was stirred at ambient temperature for 16 h then evaporated to dryness. The residue was redissolved in DCM (25 mL) and azetidine hydrochloride (1.12 g, 12.0 mmol) added followed by triethylamine (4.18 mL, 30.0 mmol). The mixture was stirred at ambient temperature for 2 h then concentrated in vacuo. The residue was partitioned between ethyl acetate and 1N hydrochloric acid, the organic phase washed with a saturated aqueous solution of sodium bicarbonate followed by brine, dried (MgSO₄), and concentrated in vacuo. The title compound was crystallized from an ethyl acetate/hexane mixture to give a white crystalline solid (1.0 g, 51%). ¹H NMR δ (CDCl₃): 2.4 (m, 2H), 4.3 (m, 4H), 7.2 (m, 1H), 7.4 (m, 1H), 7.5 (t, 1H).

### Intermediate 2: 3-Hydroxy-5-{[(1S)-1-(hydroxymethyl)propyl]oxy}-N-(1-methyl-1H-pyrazol-3-yl)benzamide

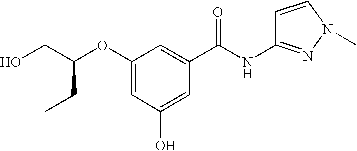 Iodotrimethylsilane (1.11 mL, 7.8 mmol) was added to a solution of 3-hydroxy-5-{[(1S)-1-(methoxymethyl)propyl]oxy}-N-(1-methyl-1H-pyrazol-3-yl)benzamide (Intermediate 3, 500 mg, 1.6 mmol) in acetonitrile (25 mL), and the resulting mixture stirred for 16 hours. Saturated sodium hydrogencarbonate solution (10 mL) was added, the solution stirred for 10 mins, saturated aqueous sodium thiosulfate (5 mL) was added then the acetonitrile was removed in vacuo. The residual aqueous layer was extracted with ethyl acetate (3×40 mL) and the organic layers combined, dried (MgSO₄), filtered and evaporated and purified by column chromatography (eluting with 85% ethyl acetate in isohexane) to give the title compound (405 mg, 83%) as a colourless foam. ¹H NMR 6 (d₆-DMSO): 0.95 (t, 3H), 1.5-1.8 (m, 2H), 3.5 (m, 2H), 3.8 (s, 3H), 4.3 (m, 1H), 4.8 (t, 1H), 6.45 (s, 1H), 6.55 (s, 1H), 6.9 (s, 1H), 7.05 (s, 1H), 7.55 (s, 1H), 9.6 (s, 1H); m/z 306 (M+H)⁺

### Intermediate 3: 3-Hydroxy-5-{[(1S)-1-(methoxymethyl)propyl]oxy}-N-(1-methyl-1H-pyrazol-3-yl)benzamide

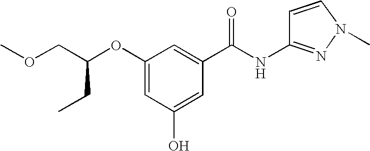 10% w/w Palladium on carbon (450 mg) was added to a solution of 3-(benzyloxy)-5-{[(1S)-1-(methoxymethyl)propyl]oxy}-N-(1-methyl-1H-pyrazol-3-yl)benzamide (Intermediate 4: 4.6 g, 11 mmol) in THF (50 mL) and methanol (50 mL) and the resulting mixture stirred under an atmosphere of hydrogen for 6 hours. The mixture was filtered and evaporated to afford the title compound as a white solid (3.6 g 100%). ¹H NMR δ (CDCl₃): 0.95 (t, 3H), 1.6-1.8 (m, 2H), 3.4 (s, 3H), 3.55 (m, 2H), 3.8 (s, 3H), 4.3 (m, 1H), 6.65 (s, 1H), 6.8 (s, 1H), 7.0 (m, 2H), 7.2 (m, 1H), 7.3 (s, 1H), 8.7 (s, 1H), m/z 320 (M+H)⁺

### Intermediate 4: 3-(Benzyloxy)-5-{[(1S)-1-(methoxymethyl)propyl]oxy}-N-(1-methyl-1H-pyrazol-3-yl)benzamide

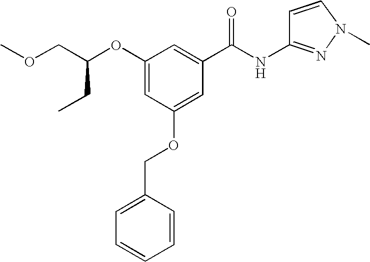 HATU (8.53 g, 22.4 mmol) was added to a solution of 3-(benzyloxy)-5-{[(1S)-1-(methoxymethyl)propyl]oxy}benzoic acid (Intermediate 5: 4.75 g, 14.4 mmol) and 3-amino-1-methyl-1H-pyrazole (2.04 g, 21 mmol) in DMF (25 mL) followed by the addition of DIPEA (7.0 mL, 40 mmol) and the resulting mixture was stirred for 16 hours. The mixture was partitioned between ethyl acetate (100 mL) and water (30 mL). The organic layer was separated, washed with 1N citric acid (30 mL), water (30 mL), saturated sodium bicarbonate (30 mL), water (30 mL) and brine (30 mL) then dried (MgSO₄) and evaporated. The residue was purified by column chromatography (eluting with 50% ethyl acetate in isohexane) to give the title compound (4.57 g, 85%) as a colourless oil ¹H NMR δ (CDCl₃): 0.95 (t, 3H), 1.6-1.8 (m, 2H), 3.4 (s, 3H), 3.55 (m, 2H), 3.8 (s, 3H), 4.3 (m, 1H), 5.05 (s, 2H), 6.75 (s, 1H), 6.8 (s, 1H), 7.05 (d, 2H), 7.25 (s, 1H), 7.4 (m, 5H), 8.45 (s, 1H), m/z 410 (M+H)⁺

### Intermediate 5: 3-(Benzyloxy)-5-{[(1S)-1-(methoxymethyl)propyl]oxy}benzoic acid

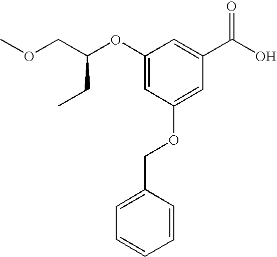 1N Lithium hydroxide solution in water (40 mL, 40 mmol) was added to a solution of methyl methyl 3-(benzyloxy)-5-{[(1S)-1-(methoxymethyl)propyl]oxy}benzoate (Intermediate 6: 6.85 g, 20 mmol) in THF (75 mL) and methanol (25 mL), then a further 100 mL water was added portionwise over 2 hours with stirring. The organic solvents were removed by evaporation and the cloudy solution filtered. The pH of the filtrate was adjusted to 3 by the addition of 2 M hydrochloric acid. This was extracted with ethyl acetate (3×70 mL). The combined organic extracts were dried (MgSO₄) and evaporated to afford the title compound as a colourless oil which solidified (6.36 g, 96%). ¹H NMR δ (CDCl₃): 0.95 (t, 3H), 1.6-1.8 (m, 2H), 3.4 (s, 3H), 3.55 (m, 2H), 4.3 (m, 1H), 5.05 (s, 2H), 6.8 (s, 1H), 7.3-7.5 (m, 7H), m/z 329 (M−H)⁻

### Intermediate 6: Methyl 3-(benzyloxy)-5-{[(1S)-1-(methoxymethyl)propyl]oxy}benzoate

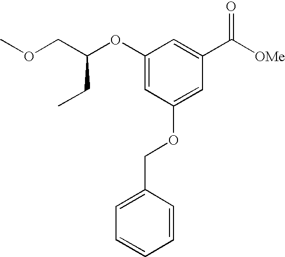 A solution of 40% DEAD in toluene (15.8 mL, 36.25 mmol) was added dropwise over 30 minutes to a stirred solution of methyl 3-(benzyloxy)-5-hydroxybenzoate (Intermediate 7, 7.5 g, 29 mmol), (R)-1-methoxy-butan-2-ol [Coke, J. L.; Shue, R. S. (1973) J. Org. Chem. 38, 2210-2211] (3.76 g, 36.25 mmol) and triphenylphosphine (9.5 g, 36.25 mmol) in dry THF (75 mL) which was cooled in an ice-bath. The reaction mixture was allowed to warm slowly to 10° C. and stirred for 16 hours. The THF was evaporated. The residue was dissolved in 30% ethyl acetate in isohexane and cooled in ice. The resultant precipitate was removed by filtration and washed with 10% ethyl acetate in isohexane. The filtrate was evaporated and purified by column chromatography (eluting with 10% ethyl acetate in isohexane) to give the title compound (6.85 g, 68%) as a colourless oil ¹H NMR δ (CDCl₃): 0.95 (t, 3H), 1.6-1.8 (m, 2H), 3.35 (s, 3H), 3.55 (m, 2H), 3.9 (s, 3H), 4.3 (m, 1H), 5.05 (s, 2H), 6.8 (s, 1H), 7.25 (m, 2H), 7.4 (m, 5H), m/z 345 (M+H)⁺

### Intermediate 7: Methyl 3-(benzyloxy)-5-hydroxybenzoate

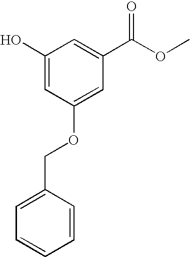 Potassium carbonate (9 mol) was added to a stirred solution of methyl 3,5-dihydroxybenzoate (5.95 mol) in DMF (6 L) and the suspension stirred at RT under argon. To this was added benzyl bromide (8.42 mol) slowly over 1 hour, with a slight exotherm, and the reaction mixture stirred overnight at ambient temperature. The reaction was quenched cautiously with ammonium chloride solution (5 L) followed by water (35 L). The aqueous suspension was extracted with DCM (1×3 L and 2×5 L). The combined extracts were washed with water (10 L) and dried overnight (MgSO₄). The solution was evaporated in vacuo, and the crude product purified by column chromatography in 3 batches (flash column, 3×2 kg silica, eluting with an increasing gradient of 10 to 100% DCM in isohexane followed by 50% ethyl acetate in DCM) to eliminate starting material. The crude eluant was purified by HPLC in 175 g batches (Amicon HPLC, 5 kg normal-phase silica, eluting with 20% ethyl acetate in isohexane) to give the title compound (21% yield); ¹H NMR δ (d₆-DMSO): 3.8 (s, 3H), 5.1 (s, 2H), 6.65 (m, 1H), 7.0 (m, 1H), 7.05 (m, 1H), 7.3-7.5 (m, 5H), 9.85 (br s, 1H)

### EXAMPLES 2-11

The following compounds were prepared in a similar manner to Example 1 by reaction of the appropriate arylfluoride with 3-hydroxy-5-{[(is)-1-(hydroxymethyl)propyl]oxy}-N-(1-methyl-1H-pyrazol-3-yl)benzamide (Example 2-9) or N-(1-ethyl-1H-pyrazol-3-yl)-3-hydroxy-5-{[(1S)-1-(hydroxymethyl)propyl]oxy}benzamide (Example 10-11).

| Example | Structure | m/z | ¹H NMR δ (CDCl₃): |
|---|---|---|---|
| 2 | 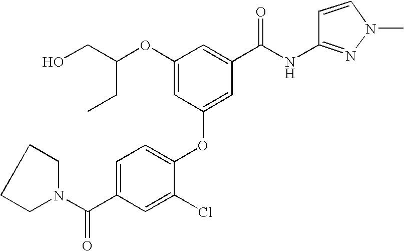 | 513 (M + H)⁺ | 0.95(t, 3H), 1.65-1.8(m, 2H), 1.85-2.05(m, 5H), 3.5(m, 2H), 3.7(m, 2H), 3.8(m, 5H), 4.3(m, 1H0, 6.75(m, 2H), 7.1(m, 2H), 7.3(m, 2H), 7.4 (d, 1H), 7.7(s, 1H), 8.6(s, 1H) |
| 3 | 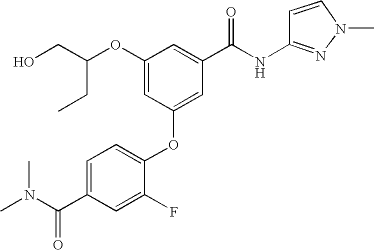 | 471 (M + H)⁺ | 0.95(t, 3H), 1.6-1.8(m, 2H), 2.0(b, 1H), 3.0(s, 6H), 3.8(m, 5H), 4.3(m, 1H), 6.8(m, 2H), 7.0-7.1(m, 2H), 7.1-7.335(m, 4H), 8.6(s, 1H) |
| 4 | 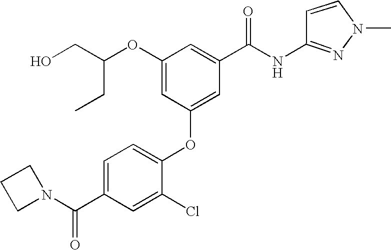 | 500 (M + H)⁺ | 0.95(t, 3H), 1.6-1.8(m, 2H), 2.2(b, 1H), 2.4(m, 2H), 3.8(m, 5H), 4.2-4.4(m, 5H), 6.8(m, 2H), 7.0-7.1(m, 2H), 7.3(m, 2H), 7.4(d, 1H), 7.5(d, 1H), 8.8(s, 1H) |
| 5 | 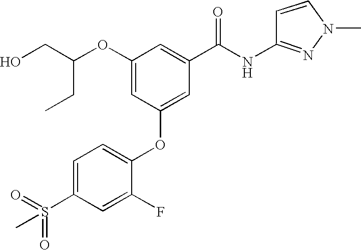 | 478 (M + H)⁺ | 0.95(t, 3H), 1.6-1.8(m, 2H), 2.1(t, 1H), 3.1(s, 3H), 3.8(m, 5H), 4.4(m, 1H), 6.75(s, 1H), 6.8 (s, 1H), 7.05(s, 1H), 7.2(t, 1H), 7.4(m, 2H), 7.7 (d, 1H), 7.8(d, 1H), 8.6(s, 1H) |
| 6 | 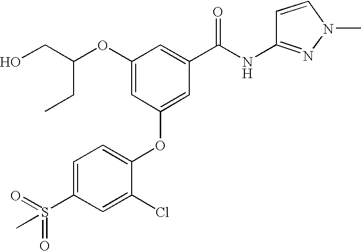 | 494 (M + H)⁺ | 0.95(t, 3H), 1.6-1.8(m, 2H), 2.1(b, 1H), 3.1(s, 3H), 3.8(m, 5H), 4.3(m, 1H), 6.75(s, 1H), 6.8 (s, 1H), 7.05(s, 1H), 7.1(s, 1H), 7.4(m, 2H), 7.8 (d, 1H), 8.1(s, 1H), 8.6(s, 1H) |
| 7 | 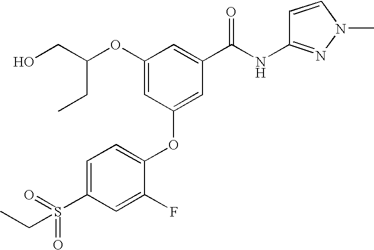 | 492 (M + H)⁺ | 0.95(t, 3H), 1.3(t, 3H), 1.6-1.8(m, 2H), 2.1(b, 1H), 3.1(m, 2H), 3.8(m, 5H), 4.3(m, 1H), 6.75 (s, 1H), 6.8(s, 1H), 7.1(s, 1H), 7.2(t, 1H), 7.4 (m, 2H), 7.7(d, 1H), 7.8(d, 1H), 8.6(b, 1H) |
| 8 | 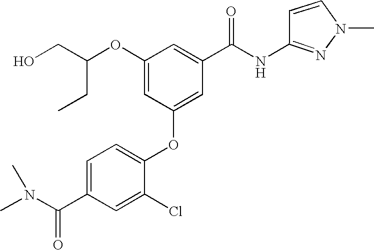 | 487 (M + H)⁺ | ¹H NMR δ CDCl₃: 0.95(t, 3H), 1.6-1.8(m, 2H), 2.0(b, 1H), 3.05(s, 6H), 3.8(m, 5H), 4.3 (m, 1H), 6.75(s, 1H), 6.8(s, 1H), 7.2-7.3(m, 2H), 7.35(d, 1H), 7.55(s, 1H), 8.6(s, 1H) |
| 9 | 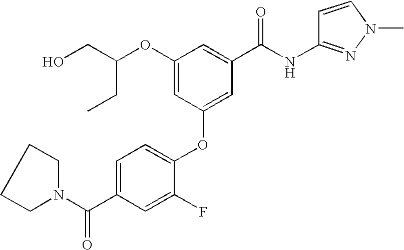 | 497 (M + H)⁺ | ¹H NMR δ (CDCl₃): 0.95(t, 3H), 1.65-1.8(m, 2H), 1.8-2.0(m, 4H), 2.1(b, 1H), 3.5(m, 2H), 3.6(m, 2H), 3.75(m, 5H), 4.3(m, 1H), 6.75(m, 2H), 7.0(s, 1H), 7.1(m, 1H), 7.2(s, 1H), 7.25 (m, 1H), 7.3(d, 1H), 7.4(d, 1H), 8.7(b, 1H) |
| 10 | 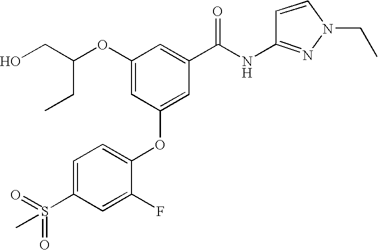 | 492 (M + H)⁺ | 0.95(t, 3H), 1.45(t, 3H), 1.6-1.8(m, 2H), 2.0(b, 1H), 3.1(s, 3H), 3.8(m, 2H), 4.05(q, 2H), 4.4 (m, 1H), 6.75(s, 1H), 6.8(s, 1H), 7.05(s, 1H), 7.2(t, 1H), 7.4(m, 2H), 7.7(d, 1H), 7.8(d, 1H), 8.65(s, 1H) |
| 11 | 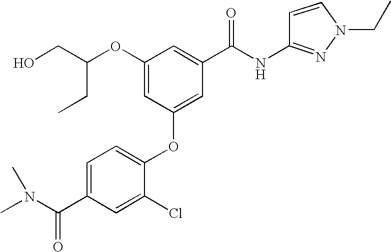 | 501 (M + H)⁺ | 0.95(t, 3H), 1.45(t, 3H), 1.6-1.8(m, 2H), 2.05 (b, 1H), 3.0(s, 6H), 3.8(m, 2H), 4.1(q, 2H), 4.35(m, 1H), 6.75(m, 2H), 7.0(m, 2H), 7.25(m, 1H), 7.3(m, 2H), 7.6(s, 1H), 8.5(s, 1H) |

The appropriate aryl fluorides for Examples 2-4, 8, 9, 11 were prepared in a similar manner to Intermediate 1 starting from the appropriate benzoic acid and amine.

### Intermediate 8: 1-(3-Chloro-4-fluorobenzoyl)azetidine

¹H NMR δ (CDCl₃): 2.4 (m, 2H), 4.2-4.4 (m, 4H), 7.2 (m, 1H), 7.55 (m, 1H), 7.7 (m, 1H)

### Intermediate 9: 3,4-Difluoro-N,N-dimethylbenzamide

¹H NMR δ (CDCl₃): 2.9-3.2 (m, 6H), 7.2 (m, 2H), 7.3 (m, 1H). m/z 186 (M+H)⁺.

### Intermediate 10: 3-Chloro-4-fluoro-N,N-dimethylbenzamide

¹H NMR 6 (d₆-DMSO): 2.90 (s, 3H), 2.96 (s, 3H), 7.42 (m, 2H), 7.62 (dd, 1H). 202, 204 (M+H)⁺.

### Intermediate 11: 1-(3,4-Difluorobenzoyl)pyrrolidine

¹H NMR δ (CDCl₃): 1.8-2.1 (m, 4H), 3.4 (t, 2H), 3.7 (t, 2H), 7.2 (m, 1H), 7.3 (m, 1H), 7.4 (t, 1H).

### Intermediate 12: 1-(3-Chloro-4-fluorobenzoyl)pyrrolidine

¹H NMR 6 (d₆-DMSO): 1.8 (m, 4H), 3.4 (t, 2H), 3.5 (t, 2H), 7.4 (t, 1H), 7.5 (m, 1H), 7.7 (d, 1H). m/z 228, 230 (M+H)⁺.

The aryl fluoride used in the preparation of example 7 was prepared as follows.

### Intermediate 13: 3,4-Difluorophenyl ethyl sulfone

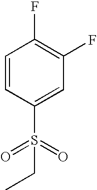 To a solution of 4-ethylsulphanyl-1,2-difluorobenzene (1.50 g) in DCM (50 mL) was added 75% m-chloroperbenzoic acid (2.97 g) and the mixture stirred at ambient temperature for 16 h. The mixture was washed successively with saturated potassium carbonate (20 mL) and brine (30 mL) then dried with magnesium sulphate, filtered and reduced in vacuo. The resultant clear oil was chromatographed on silica (eluting with 0-50% ethyl acetate in iso-hexane) and the faster running product isolated (0.90 g). The required 3,4-difluorophenyl ethyl sulfone was used without further characterisation.
The aryl fluorides used in the preparation of Examples 5, 6 and 10 were prepared in an analogous fashion to Intermediate 13.

### Intermediate 14: 3,4-Difluorophenyl methyl sulphone

¹H NMR δ (CDCl₃): 3.05 (s, 3H), 7.2 (q, 1H), 7.7-7.8 (m, 2H)

### Intermediate 15: 3-Chloro-4-Fluorophenyl methyl sulphone

¹H NMR δ (CDCl₃): 3.1 (s, 3H), 7.3 (t, 1H), 7.35 (m, 1H), 8.0 (dd, 1H)

The starting N-(1-ethyl-1H-pyrazol-3-yl)-3-hydroxy-5-{[(1S)-1-(hydroxymethyl)propyl]oxy}benzamide (Intermediate 16) used in Examples 10 and 11 was prepared in the same way as Intermediate 2 starting from 3-(benzyloxy)-5-{[(1S)-1-(methoxymethyl)propyl]oxy}benzoic acid (Intermediate 5) and using 3-amino-1-ethyl-1H-pyrazole [Giller S. A., Eremeev A. V., Kalvin'sh I. Ya., Liepin'sh E. E., Tikhomirov D. A., Chem. Heterocycl. Compd. (Engl. Transl.), 11, 1975, 212] in place of the 3-amino-1-methyl-1H-pyrazole.

### Intermediate 16: N-(1-Ethyl-1H-pyrazol-3-yl)-3-hydroxy-5-{[(1S)-1-(hydroxymethyl)propyl]oxy}benzamide

¹H NMR δ (d₆-DMSO): 0.95 (t, 3H), 1.35 (t, 3H), 1.5-1.8 (m, 2H), 3.5 (m, 2H), 4.1 (q, 2H), 4.3 (m, 1H), 4.75 (t, 1H), 6.45 (s, 1H), 6.55 (s, 1H), 6.9 (s, 1H), 7.05 (s, 1H), 7.6 (s, 1H), 9.6 (s, 1H), 10.6 (b, 1H); m/z 320 (M+H)⁺

### Intermediate 17: N-(1-Ethyl-1H-pyrazol-3-yl)-3-hydroxy-5-{[(1S)-1-(methoxymethyl)propyl]oxy}benzamide

¹H NMR δ (CDCl₃): 0.95 (t, 3H), 1.5 (t, 3H), 1.6-1.8 (m, 2H), 3.35 (s, 3H), 3.55 (m, 2H), 4.1 (q, 3H), 6.6 (s, 1H), 6.8 (s, 1H), 7.0 (m, 2H), 7.2 (s, 1H), 8.6 (s, 1H), m/z 334 (M+H)⁺

### Intermediate 18: 3-(Benzyloxy)-N-(1-ethyl-1H-pyrazol-3-yl)-5-{[(1S)-1-(methoxymethyl)propyl]oxy}benzamide

¹H NMR δ (CDCl₃): 0.95 (t, 3H), 1.5 (t, 3H), 1.6-1.8 (m, 2H), 3.35 (s, 3H), 3.55 (m, 2H), 4.1 (q, 3H), 4.35 (m, 1H), 5.05 (s, 2H), 6.75 (s, 1H), 6.8 (s, 1H), 7.05 (s, 1H), 7.1 (s, 1H), 7.3 7.4 (m, 6H), 8.4 (s, 1H), m/z 424 (M+H)⁺

### EXAMPLE 12

### 3-{[(1S)-1-(Hydroxymethyl)propyl]oxy}-N-(1-methyl-1H-pyrazol-3-yl)-5-[4-(pyrrolidin-1-ylcarbonyl)phenoxy]benzamide

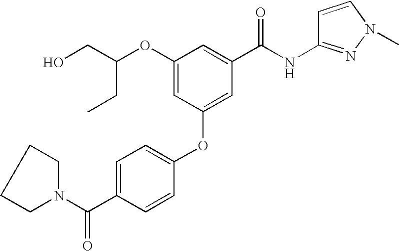 10% w/w Palladium on carbon (50 mg) was added to a solution of 3-[2-chloro-4-(pyrrolidin-1-ylcarbonyl)phenoxy]-5-{[(1S)-1-(hydroxymethyl)propyl]oxy}-N-(1-methyl-1H-pyrazol-3-yl)benzamide (Example 2, 159 mg, 0.31 mmol) in a mixture of THF (3 mL), methanol (3 mL) and ethylacetate (5 mL) containing triethylamine (0.257 mL, 1.86 mmol) and the resulting mixture stirred under an atmosphere of hydrogen for 48 hours. The mixture was filtered and evaporated to dryness. The residue was taken up in ethyl acetate (20 mL), washed with 0.5N HCl (5 mL), brine (5 mL), dried (MgSO₄), and evaporated to afford the title compound as a white foam (112 mg 76%). ¹H NMR δ (CDCl₃): 0.95 (t, 3H), 1.65-1.8 (m, 2H), 1.85-2.0 (m, 4H), 3.5 (m, 2H), 3.7 (m, 2H), 3.8 (m, 5H), 4.3 (m, 1H), 6.75 (s, 2H), 6.95-7.1 (m, 3H), 7.25 (m, 2H), 7.55 (d, 2H), 8.6 (s, 1H); m/z 479 (M+H)⁺

### EXAMPLES 13-15

The following examples were prepared in a similar manner to Example 12 from the appropriate intermediates indicated below.

| Example | SM | Structure | m/z | ¹H NMR δ (CDCl₃): |
|---|---|---|---|---|
| 13 | Eg 4 | 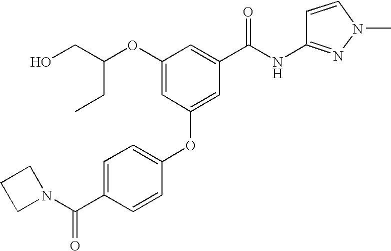 | 465 (M + H)⁺ | 0.95(t, 3H), 1.6-1.8(m, 2H), 2.1(b, 1H), 2.35 (m, 2H), 3.8(m, 5H), 4.2-4.4(m, 5H), 6.8(m, 2H), 7.0(d, 2H), 7.1(s, 1H), 7.25(m, 2H), 7.6 (d, 2H), 8.9(s, 1H) |
| 14 | Eg 11 | 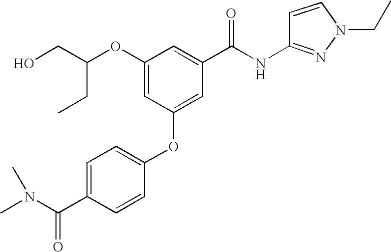 | 467 (M + H)⁺ | 0.95(t, 3H), 1.4(m, 3H), 1.6-1.8(m, 2H), 2.05(b, 1H), 3.0(s, 6H), 3.8(m, 2H), 4.1(q, 2H), 4.35(m, 1H), 6.75(m, 2H), 7.05(d, 2H), 7.1(s, 1H), 7.25(s, 1H), 7.3(s, 1H), 7.4(d, 2H), 8.6(s, 1H) |
| 15 | Eg 8 | 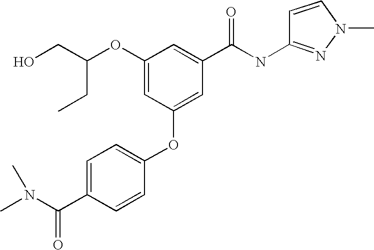 | 453 (M + H)⁺ | 0.95(t, 3H), 1.7(m, 2H), 2.1(b, 1H), 3.1(s, 6H), 3.75(m, 5H), 4.35(m, 1H), 6.75(m, 2H), 6.95-7.1(m, 3H), 7.25(m, 2H), 7.45(d, 2H), 8.6(s, 1H) |

Biological
Tests:
The biological effects of the compounds of formula (I) may be tested in the following way:
(1) Enzymatic Activity

Enzymatic activity of recombinant human pancreatic GLK may be measured by incubating GLK, ATP and glucose. The rate of product formation may be determined by coupling the assay to a G-6-P dehydrogenase, NADP/NADPH system and measuring the linear increase with time of the optical density at 340 nm (Matschinsky et al 1993). Activation of GLK by compounds can be assessed using this assay in the presence or absence of GLKRP as described in Brocklehurst et al (Diabetes 2004, 53, 535-541).

Production of Recombinant GLK and GLKRP:

Human GLK and GLKRP cDNA was obtained by PCR from human pancreatic and hepatic mRNA respectively, using established techniques described in Sambrook J, Fritsch EF & Maniatis T, 1989. PCR primers were designed according to the GLK and GLKRP cDNA sequences shown in Tanizawa et al 1991 and Bonthron, D. T. et al 1994 (later corrected in Warner, J. P. 1995).

Cloning in Bluescript II Vectors

GLK and GLKRP cDNA was cloned in E. coli using pBluescript II, (Short et al 1998) a recombinant cloning vector system similar to that employed by Yanisch-Perron C et al (1985), comprising a colEI-based replicon bearing a polylinker DNA fragment containing multiple unique restriction sites, flanked by bacteriophage T3 and T7 promoter sequences; a filamentous phage origin of replication and an ampicillin drug resistance marker gene.

Transformations

E. Coli transformations were generally carried out by electroporation. 400 mL cultures of strains DH5a or BL21(DE3) were grown in L-broth to an OD 600 of 0.5 and harvested by centrifugation at 2,000 g. The cells were washed twice in ice-cold deionised water, resuspended in 1 mL 10% glycerol and stored in aliquots at −70° C. Ligation mixes were desalted using Millipore V series™ membranes (0.0025 mm) pore size). 40 mL of cells were incubated with 1 mL of ligation mix or plasmid DNA on ice for 10 minutes in 0.2 cm electroporation cuvettes, and then pulsed using a Gene Pulser™ apparatus (BioRad) at 0.5 kVcm⁻¹, 250 mF. Transformants were selected on L-agar supplemented with tetracyline at 10 mg/mL or ampicillin at 100 mg/mL.

Expression

GLK was expressed from the vector pTB375NBSE in E. coli BL21 cells, producing a recombinant protein containing a 6-His tag immediately adjacent to the N-terminal methionine. Alternatively, another suitable vector is pET21(+)DNA, Novagen, Cat number 697703. The 6-His tag was used to allow purification of the recombinant protein on a column packed with nickel-nitrilotriacetic acid agarose purchased from Qiagen (cat no 30250).

GLKRP was expressed from the vector pFLAG CTC (IBI Kodak) in E. coli BL21 cells, producing a recombinant protein containing a C-terminal FLAG tag. The protein was purified initially by DEAE Sepharose ion exchange followed by utilisation of the FLAG tag for final purification on an M2 anti-FLAG immunoaffinity column purchased from Sigma-Aldrich (cat no. A1205).

(2) Oral Glucose Tolerance Test (OGTT)

Oral glucose tolerance tests were done on conscious Zucker obese fa/fa rats (age 12-13 weeks or older) fed a high fat diet (45% kcal fat) for at least two weeks prior to experimentation. The animals were fasted for 2 hours before use for experiments. A test compound or a vehicle was given orally 120 minutes before oral administration of a glucose solution at a dose of 2 g/kg body weight. Blood glucose levels were measured using a Accucheck glucometer from tail bled samples taken at different time points before and after administration of glucose (time course of 60 minutes). A time curve of the blood glucose levels was generated and the area-under-the-curve (AUC) for 120 minutes was calculated (the time of glucose administration being time zero). Percent inhibition was determined using the AUC in the vehicle-control group as zero percent inhibition.

3) Measurements of Plasma Protein Binding of Compounds

The plasma protein binding of compounds was measured using the equilibrium dialysis technique (W. Lindner et al, J. Chromatography, 1996, 677, 1-28). Compound was dialysed at a concentration of 20 μM for 18 hours at 37° C. with plasma and isotonic phosphate buffer pH 7.4 (1 ml of each in the dialysis cell). A Spectrum® 20-cell equilibrium dialyser was used together with Teflon, semi-micro dialysis cells and Spectra/Por®2 membrane discs with a molecular weight cut off 12-14000 Dalton, 47 mm (supplied by PerBio Science UK Ltd, Tattenhall, Cheshire). Plasma and buffer samples are removed following dialysis and analysed using HPLCUV/MS (high performance liquid chromatography with UV and mass spec detection) to give the % free level in plasma.

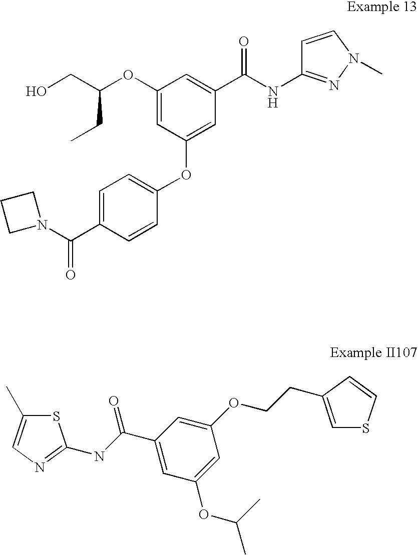

Compounds of the invention generally have an activating activity for glucokinase with an EC₅₀ of less than about 500 nM, preferably less than about 50 nm.

For example, Example 13 has an EC₅₀ of 0.02 μm.

Example II107 in WO 03/015774 has an EC₅₀ of 0.15 μm.

Example 13 is 2% free in rat plasma.

### REFERENCES

1 Printz, R. L., Magnuson, M. A. and Granner, D. K. (1993) Annual Review of Nutrition 13, 463-96
2 DeFronzo, R. A. (1988) Diabetes 37, 667-87
3 Froguel, P., Zouali, H., Vionnet, N., Velho, G., Vaxillaire, M., Sun, F., Lesage, S., Stoffel, M., Takeda, J. and Passa, P. (1993) New England Journal of Medicine 328, 697-702
4 Bell, G. I., Pilkis, S. J., Weber, I. T. and Polonsky, K. S. (1996) Annual Review of Physiology 58, 171-86
5. Velho, G., Petersen, K. F., Perseghin, G., Hwang, J. H., Rothman, D. L., Pueyo, M. E., Cline, G. W., Froguel, P. and Shulman, G. I. (1996) Journal of Clinical Investigation 98, 1755-61
6 Christesen, H. B., Jacobsen, B. B., Odili, S., Buettger, C., Cuesta-Munoz, A., Hansen, T., Brusgaard, K., Massa, O., Magnuson, M. A., Shiota, C., Matschinsky, F. M. and Barbetti, F. (2002) Diabetes 51, 1240-6
6a Gloyn, A. L., Noordam, K., Willemsen, M. A. A. P., Ellard, S., Lam, W. W. K., Campbell, I. W., Midgley, P., Shiota, C., Buettger, C., Magnuson, M. A., Matschinsky, F. M., and Hattersley, A. T.; Diabetes 52: 2433-2440
7 Glaser, B., Kesavan, P., Heyman, M., Davis, E., Cuesta, A., Buchs, A., Stanley, C. A., Thornton, P. S., Permutt, M. A., Matschinsky, F. M. and Herold, K. C. (1998) New England Journal of Medicine 338, 226-30
8 Caro, J. F., Triester, S., Patel, V. K., Tapscott, E. B., Frazier, N. L. and Dohm, G. L.
   (1995) Hormone & Metabolic Research 27, 19-22
9 Desai, U. J., Slosberg, E. D., Boettcher, B. R., Caplan, S. L., Fanelli, B., Stephan, Z., Gunther, V. J., Kaleko, M. and Connelly, S. (2001) Diabetes 50, 2287-95
10 Shiota, M., Postic, C., Fujimoto, Y., Jetton, T. L., Dixon, K., Pan, D., Grimsby, J., Grippo, J. F., Magnuson, M. A. and Chemington, A. D. (2001) Diabetes 50, 622-9
11 Ferre, T., Pujol, A., Riu, E., Bosch, F. and Valera, A. (1996) Proceedings of the National Academy of Sciences of the United States of America 93, 7225-30
12 Seoane, J., Barbera, A., Telemaque-Potts, S., Newgard, C. B. and Guinovart, J. J. (1999) Journal of Biological Chemistry 274, 31833-8
13 Moore, M. C., Davis, S, N., Mann, S. L. and Chemington, A. D. (2001) Diabetes Care 24, 1882-7
14 Alvarez, E., Roncero, I., Chowen, J. A., Vazquez, P. and Blazquez, E. (2002) Journal of Neurochemistry 80, 45-53
15 Lynch, R. M., Tompkins, L. S., Brooks, H. L., Dunn-Meynell, A. A. and Levin, B. E.
   (2000) Diabetes 49, 693-700
16 Roncero, I., Alvarez, E., Vazquez, P. and Blazquez, E. (2000) Journal of Neurochemistry 74, 1848-57
17 Yang, X. J., Kow, L. M., Funabashi, T. and Mobbs, C. V. (1999) Diabetes 48, 1763-1772
18 Schuit, F. C., Huypens, P., Heimberg, H. and Pipeleers, D. G. (2001) Diabetes 50, 1-11
19 Levin, B. E. (2001) International Journal of Obesity 25, supplement 5, S68-S72.
20 Alvarez, E., Roncero, I., Chowen, J. A., Thorens, B. and Blazquez, E. (1996) Journal of Neurochemistry 66, 920-7
21 Mobbs, C. V., Kow, L. M. and Yang, X. J. (2001) American Journal of Physiology—Endocrinology & Metabolism 281, E649-54
22 Levin, B. E., Dunn-Meynell, A. A. and Routh, V. H. (1999) American Journal of Physiology 276, R1223-31
23 Spanswick, D., Smith, M. A., Groppi, V. E., Logan, S. D. and Ashford, M. L. (1997) Nature 390, 521-5
24 Spanswick, D., Smith, M. A., Mirshamsi, S., Routh, V. H. and Ashford, M. L. (2000) Nature Neuroscience 3, 757-8
25 Levin, B. E. and Dunn-Meynell, A. A. (1997) Brain Research 776, 146-53
26 Levin, B. E., Govek, E. K. and Dunn-Meynell, A. A. (1998) Brain Research 808, 317-9
27 Levin, B. E., Brown, K. L. and Dunn-Meynell, A. A. (1996) Brain Research 739, 293-300
28 Rowe, I. C., Boden, P. R. and Ashford, M. L. (1996) Journal of Physiology 497, 365-77
29 Fujimoto, K., Sakata, T., Arase, K., Kurata, K., Okabe, Y. and Shiraishi, T. (1985) Life Sciences 37, 2475-82
30 Kurata, K., Fujimoto, K. and Sakata, T. (1989) Metabolism: Clinical & Experimental 38, 46-51
31 Kurata, K., Fujimoto, K., Sakata, T., Etou, H. and Fukagawa, K. (1986) Physiology & Behavior 37, 615-20
32 Jetton T. L., Liang Y., Pettepher C. C., Zimmerman E. C., Cox F. G., Horvath K., Matschinsky F. M., and Magnuson M. A., J. Biol. Chem., February 1994; 269: 3641-3654.
33 Reimann F. and Gribble F. M., Diabetes 2002 51: 2757-2763
34 Cheung A. T., Dayanandan B., Lewis J. T., Korbutt G. S., Rajotte R. V., Bryer-Ash M., Boylan M. O., Wolfe M. M., Kieffer T. J., Science, Vol 290, Issue 5498, 1959-1962, 8 Dec. 2000

1. A compound of Formula (I): 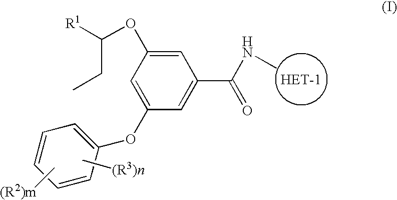
wherein:
R¹ is hydroxymethyl;
R² is selected from —C(O)NR⁴R⁵, —SO₂NR⁴R⁵, —S(O)ₚR⁴ and HET-2;
HET-1 is a 5- or 6-membered, C-linked heteroaryl ring containing a nitrogen atom in the 2-position and optionally 1 or 2 further ring heteroatoms independently selected from O, N and S; which ring is optionally substituted on an available carbon atom, or on a ring nitrogen atom provided it is not thereby quaternised, with 1 or 2 substituents independently selected from R⁶;
HET-2 is a 4-, 5- or 6-membered, C- or N-linked heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to a S(O) or S(O)₂ group, which ring is optionally substituted on an available carbon or nitrogen atom by 1 or 2 substituents independently selected from R⁷;
R³ is selected from halo, fluoromethyl, difluoromethyl, trifluoromethyl, methyl, methoxy and cyano;
R⁴ is selected from hydrogen, (1-4C)alkyl [optionally substituted by 1 or 2 substituents independently selected from HET-2, —OR⁵, —SO₂R⁵, (3-6C)cycloalkyl (optionally substituted with 1 group selected from R⁷) and —C(O)NR⁵R⁵], (3-6C)cycloalkyl (optionally substituted with 1 group selected from R⁷) and HET-2;
R⁵ is hydrogen or (1-4C)alkyl;
or R⁴ and R⁵ together with the nitrogen atom to which they are attached may form a heterocyclyl ring system as defined by HET-3;
R⁶ is independently selected from (1-4C)alkyl, halo, hydroxy(1-4C)alkyl, (1-4C)alkoxy(1-4C)alkyl, (1-4C)alkylS(O)p(1-4C)alkyl, amino(1-4C)alkyl, (1-4C)alkylamino(1-4C)alkyl, di(1-4C)alkylamino(1-4C)alkyl and HET-4;
R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
HET-3 is an N-linked, 4 to 6 membered, saturated or partially unsaturated heterocyclyl ring, optionally containing 1 or 2 further heteroatoms (in addition to the linking N atom) independently selected from O, N and S, wherein a —CH₂— group can optionally be replaced by a —C(O)— and wherein a sulphur atom in the ring may optionally be oxidised to a S(O) or S(O)₂ group; which ring is optionally substituted on an available carbon or nitrogen atom by 1 or 2 substituents independently selected from R⁸; or
HET-3 is an N-linked, 7 membered, saturated or partially unsaturated heterocyclyl ring, optionally containing 1 further heteroatom (in addition to the linking N atom) independently selected from O, S and N, wherein a —CH₂— group can optionally be replaced by a —C(O)— group and wherein a sulphur atom in the ring may optionally be oxidised to a S(O) or S(O)₂ group; which ring is optionally substituted on an available carbon or nitrogen atom by 1 or 2 substituents independently selected from R⁸; or
HET-3 is an 6-10 membered bicyclic saturated or partially unsaturated heterocyclyl ring, optionally containing 1 further nitrogen atom (in addition to the linking N atom), wherein a —CH₂— group can optionally be replaced by a —C(O)—; which ring is optionally substituted on an available carbon or nitrogen atom by 1 substituent selected from hydroxy (not on nitrogen) and R³;
R⁸ is selected from —OR⁵, (1-4C)alkyl, —C(O)(1-4C)alkyl, —C(O)NR⁴R⁵, (1-4C)alkylamino, di(1-4C)alkylamino, HET-3 (wherein said ring is unsubstituted), (1-4C)alkoxy(1-4C)alkyl, hydroxy(1-4C)alkyl and —S(O)pR⁵;
HET-4 is a 5- or 6-membered, C- or N-linked unsubstituted heteroaryl ring containing 1, 2 or 3 ring heteroatoms independently selected from O, N and S;
p is (independently at each occurrence) 0, 1 or 2;
m is 0 or 1;
n is 0, 1 or 2;
provided that when m is 0, then n is 1 or 2;
or a salt, pro-drug or solvate thereof.

2. A compound of the formula (I) as claimed in claim 1 or a salt, pro-drug or solvate thereof with the proviso that compounds exemplified in WO2004/076420, which would otherwise fall within the scope of this invention, are excluded.

3. A compound of the formula (I) as claimed in claim 1 or claim 2 or a salt, pro-drug or solvate thereof wherein R¹ has the (S) configuration.

4. A compound of the formula (I) as claimed in claim 1, claim 2, or claim 3 or a salt, pro-drug or solvate thereof, wherein HET-1 is a 5-membered ring.

5. A compound of the formula (I) as claimed in any one of claims 1 to 4 or a salt, pro-drug or solvate thereof, wherein R² is selected from —C(O)NR⁴R⁵ and —SO₂NR⁴R⁵ and R⁴ and R⁵ together with the nitrogen atom to which they are attached form a heterocyclyl ring system as defined by HET-3.

6. A compound of the formula (I) as claimed in any one of claims 1 to 5, or a salt, pro-drug or solvate thereof, wherein HET-3 is a 4- to 6-membered ring.

7. A compound of the formula (I) as claimed in claim 4, or a salt, pro-drug or solvate thereof, wherein R² is selected from —C(O)NR⁴R⁵ and —SO₂NR⁴R⁵ and R⁴ is selected from (1-4C)alkyl [substituted by 1 or 2 substituents independently selected from HET-2, —OR⁵, —SO₂R⁵, (3-6C)cycloalkyl (optionally substituted with 1 group selected from R⁷) and —C(O)NR⁵R⁵], (3-6C)cycloalkyl (optionally substituted with 1 group selected from R⁷) and HET-2.

8. A compound of the formula (I) as claimed in any one of claims 1 to 4, or a salt, pro-drug or solvate thereof, wherein R² is —SO₂R⁴ and R⁴ is selected from (1-4C)alkyl [substituted by 1 or 2 substituents independently selected from HET-2, —OR⁵, —SO₂R⁵, (3-6C)cycloalkyl (optionally substituted with 1 group selected from R⁷) and —C(O)NR⁵R⁵], (3-6C)cycloalkyl (optionally substituted with 1 group selected from R⁷) and HET-2.

9. A compound of the formula (I) as claimed in any one of claims 1 to 4, or a salt, pro-drug or solvate thereof, wherein R² is HET-2.

10. A compound of formula (I) as claimed in claim 1, which is one or more of the following compounds:
3-[4-(azetidin-1-ylcarbonyl)-2-fluorophenoxy]-5-{[(1S)-1-(hydroxymethyl)propyl]oxy}-N-(1-methyl-1H-pyrazol-3-yl)benzamide;
3-[2-chloro-4-(pyrrolidin-1-ylcarbonyl)phenoxy]-5-{[(1S)-1-(hydroxymethyl)propyl]oxy}-3-N-(1-methyl-1H-pyrazol-3-yl)benzamide;
3-fluoro-4-(3-{[(1S)-1-(hydroxymethyl)propyl]oxy}-5-{[(1-methyl-1H-pyrazol-3-yl)amino]carbonyl}phenoxy)-N,N-dimethylbenzamide;
3-[4-(azetidin-1-ylcarbonyl)-2-chlorophenoxy]-5-{[(1S)-1-(hydroxymethyl)propyl]oxy}-N-(1-methyl-1H-pyrazol-3-yl)benzamide;
3-[2-fluoro-4-(methylsulfonyl)phenoxy]-5-{[(1S)-1-(hydroxymethyl)propyl]oxy}-N-(1-methyl-1H-pyrazol-3-yl)benzamide;
3-[2-chloro-4-(methylsulfonyl)phenoxy]-5-{[(1S)-1-(hydroxymethyl)propyl]oxy}-N-(1-methyl-1H-pyrazol-3-yl)benzamide;
3-[4-(ethylsulfonyl)-2-fluorophenoxy]-5-{[(1S)-1-(hydroxymethyl)propyl]oxy}-N-(1-methyl-1H-pyrazol-3-yl)benzamide;
3-chloro-4-(3-{[(1S)-1-(hydroxymethyl)propyl]oxy}-5-{[(1-methyl-1H-pyrazol-3-yl)amino]carbonyl}phenoxy)-N,N-dimethylbenzamide;
3-[2-fluoro-4-(pyrrolidin-1-ylcarbonyl)phenoxy]-5-{[(1S)-1-(hydroxymethyl)propyl]oxy}-N-(1-methyl-1H-pyrazol-3-yl)benzamide;
N-(1-ethyl-1H-pyrazol-3-yl)-3-[2-fluoro-4-(methylsulfonyl)phenoxy]-5-{[(1S)-1-(hydroxymethyl)propyl]oxy}benzamide;
3-chloro-4-(3-{[(1-ethyl-1H-pyrazol-3-yl)amino]carbonyl}-5-{[(1S)-1-(hydroxymethyl)propyl]oxy}phenoxy)-N,N-dimethylbenzamide;
3-{[(1S)-1-(hydroxymethyl)propyl]oxy}-N-(1-methyl-1H-pyrazol-3-yl)-5-[4-(pyrrolidin-1-ylcarbonyl)phenoxy]benzamide;
3-[4-(azetidin-1-ylcarbonyl)phenoxy]-5-{[(1S)-1-(hydroxymethyl)propyl]oxy}-N-(1-methyl-1H-pyrazol-3-yl)benzamide;
3-{4-[(dimethylamino)carbonyl]phenoxy}-N-(1-ethyl-1H-pyrazol-3-yl)-5-{[(1S)-1-(hydroxymethyl)propyl]oxy}benzamide; and
3-{4-[(dimethylamino)carbonyl]phenoxy}-5-{[(1S)-1-(hydroxymethyl)propyl]oxy}-N-(1-methyl-1H-pyrazol-3-yl)benzamide;
or a salt, pro-drug or solvate thereof.

11. A compound of formula (I) as claimed in claim 10, which is one or more of the following compounds:
3-[4-(azetidin-1-ylcarbonyl)-2-fluorophenoxy]-5-{[(1S)-1-(hydroxymethyl)propyl]oxy}-N-(1-methyl-1H-pyrazol-3-yl)benzamide;
3-[2-chloro-4-(pyrrolidin-1-ylcarbonyl)phenoxy]-5-{[(1S)-1-(hydroxymethyl)propyl]oxy}-N-(1-methyl-1H-pyrazol-3-yl)benzamide;
3-[4-(azetidin-1-ylcarbonyl)-2-chlorophenoxy]-5-{[(1S)-1-(hydroxymethyl)propyl]oxy}-N-(1-methyl-1H-pyrazol-3-yl)benzamide;
3-[2-fluoro-4-(pyrrolidin-1-ylcarbonyl)phenoxy]-5-{[(1S)-1-(hydroxymethyl)propyl]oxy}-N-(1-methyl-1H-pyrazol-3-yl)benzamide;
3-{[(1S)-1-(hydroxymethyl)propyl]oxy}-N-(1-methyl-1H-pyrazol-3-yl)-5-[4-(pyrrolidin-1-ylcarbonyl)phenoxy]benzamide; and
3-[4-(azetidin-1-ylcarbonyl)phenoxy]-5-{[(1S)-1-(hydroxymethyl)propyl]oxy}-N-(1-methyl-1H-pyrazol-3-yl)benzamide;
or a salt, pro-drug or solvate thereof.

12. A pharmaceutical composition comprising a compound according to any one of claims 1 to 11, or a salt, pro-drug or solvate thereof, together with a pharmaceutically acceptable diluent or carrier.

13. A compound according to any one of claims 1 to 11 or a pharmaceutically-acceptable salt, solvate or pro-drug thereof for use as a medicament.

14. Use of a compound according to any one of claims 1 to 11 or a pharmaceutically-acceptable salt, solvate or pro-drug thereof in the preparation of a medicament for treatment of a disease mediated through GLK.

15. Use of a compound according to any one of claims 1 to 11 or a pharmaceutically-acceptable salt, solvate or pro-drug thereof in the preparation of a medicament for treatment of type 2 diabetes.

16. A method of treating GLK mediated diseases by administering an effective amount of a compound of Formula (I) as claimed in any one of claims 1 to 11 or salt, solvate or pro-drug thereof, to a mammal in need of such treatment.

17. The method of claim 16 wherein the GLK mediated disease is type 2 diabetes.

18. A process for the preparation of a compound of Formula (I) as claimed in any one of claims 1 to 11, which comprises a process a) to e) (wherein the variables are as defined for compounds of Formula (I) in claim 1 unless otherwise stated):
(a) reaction of an acid of Formula (III) or activated derivative thereof with a compound of Formula (IV), wherein R¹ is hydroxymethyl or a protected version thereof; 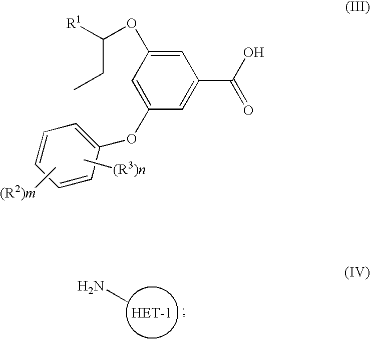
or
(b) reaction of a compound of Formula (V) with a compound of Formula (VI), 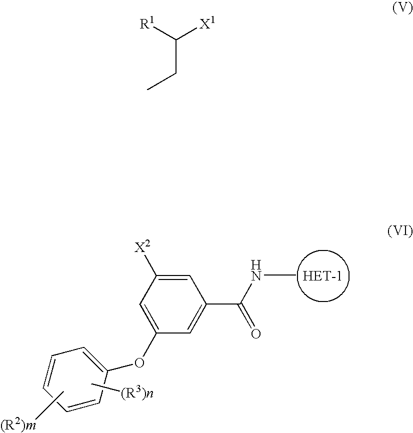
wherein X¹ is a leaving group and X² is a hydroxyl group or X¹ is a hydroxyl group and X² is a leaving group, and wherein R¹ is hydroxymethyl or a protected version thereof;
[or by reaction with the intermediate ester Formula (VII), wherein P¹ is a protecting group followed by ester hydrolysis and amide formation]; 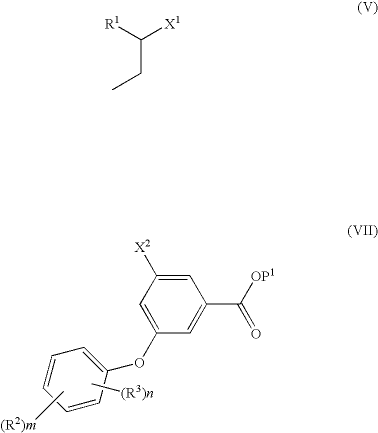
or
(c) reaction of a compound of Formula (VIII) with a compound of Formula (IX) 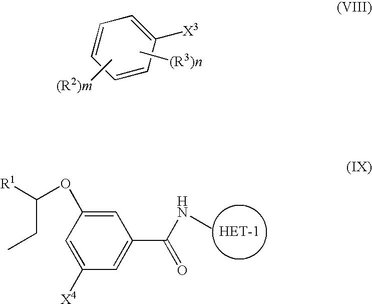
wherein X³ is a leaving group or an organometallic reagent and X⁴ is a hydroxyl group or X³ is a hydroxyl group and X⁴ is a leaving group or an organometallic reagent, and wherein R¹ is hydroxymethyl or a protected version thereof;
[or by reaction or (VIII) with the intermediate ester Formula (X), followed by ester hydrolysis and amide formation]; 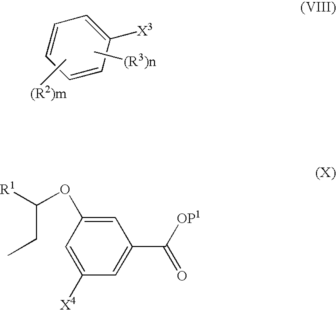
or
(d) reaction of a compound of Formula (XI) with a compound of Formula (XII), 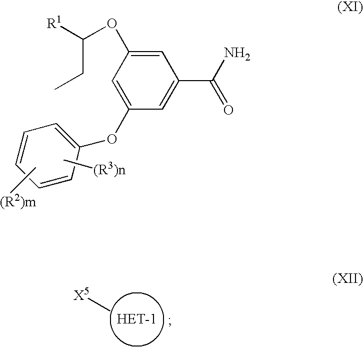
wherein X⁵ is a leaving group, and wherein R¹ is hydroxymethyl or a protected version thereof; or
e) reaction of a compound of formula (XIII) 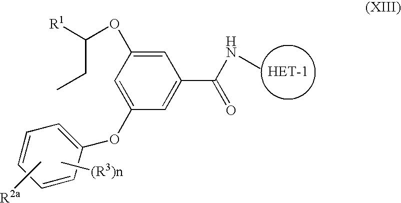
wherein R^{2a} is a precursor to R², such as a carboxylic acid, ester or anhydride (for R²—CONR⁴R⁵) or the sulfonic acid equivalents (for R² is —SO₂ NR⁴R⁵); with an amine of formula —NR⁴R⁵;
and thereafter, if necessary:
i) converting a compound of Formula (I) into another compound of Formula (I);
ii) removing any protecting groups; and/or
iii) forming a salt, pro-drug or solvate.
